(19) United States
(12) Patent Application Publication
(10) Pub. No.: US 2022/0273813 A1
(43) Pub. Date: Sep. 1, 2022

(54) PHOTOSENSITIZER-CONJUGATED ANTIMICROBIAL CELLULOSE NANOCRYSTALS AND METHODS OF SYNTHESIZING AND USING SAME

(71) Applicant: HNU MATERIALS INC., Calgary (CA)

(72) Inventors: Belinda HEYNE, Calgary (CA); David J. PRESS, Calgary (CA); Julie GROIZELEAU, Calgary (CA); Adrian Jordan Cooper TAKADA, Calgary (CA); Todd C. Sutherland, Calgary (CA); Joe HARRISON, Calgary (CA)

(21) Appl. No.: 17/638,755

(22) PCT Filed: Aug. 26, 2020
(86) PCT No.: PCT/CA2020/051163

§ 371 Date: Feb. 25, 2022

Related U.S. Application Data
(60) Provisional application No. 62/894,347, filed on Aug. 30, 2019.

(51) Int. Cl. A61K 47/69; A61K 41/00; A01N 59/00; A01N 43/84; A01N 43/60

(52) U.S. Cl. CPC A61K 47/6939; A61K 41/0057; A01N 59/00; A01N 43/84; A01N 43/60

(57) ABSTRACT

This application relates to compositions comprising cellulose nanocrystals (CNCs) and photosensitizer molecules conjugated to the CNCs. In some embodiments the compositions generate reactive oxygen when exposed to light. Methods of preparing the compositions and using the compositions or formulations containing the compositions as biocidal disinfectants are also described. In some embodiment the photosensitizer molecules comprise cationic dyes with acidic protons, such as azure A. In some embodiments the CNC and photosensitizer molecules are coupled together using a pH mediated synthetic protocol whereby the photosensitizer molecules are first dispersed in an acidic suspension of oxidized CNCs and the suspension is then adjusted to an alkaline pH to facilitate photosensitizer fixation. In some embodiments, photobactericidal potency of the composition is significantly more toxic to a broad spectrum of bacteria than the light-activated photosensitizer molecules in a non-conjugated free form. The compositions can be incorporated in various formulations for photobiocidal applications, including aqueous solutions, film-forming polymers such as paints, and hydrogels.

### TECHNICAL FIELD

This application relates to photosensitizer-conjugated antimicrobial cellulose nanocrystals and methods of synthesizing and using same.

### BACKGROUND

As flagged by the World Health Organization, antimicrobial resistance endangers populations everywhere in the world.¹ Antimicrobial resistance is the ability of a microorganism to withstand any substance of natural, semisynthetic or synthetic origin that kills or inhibits its growth. This issue affects not only people, but also animals and our environment.^{1,2} The misuse of antimicrobials coupled with the inherent ability of pathogenic bacteria to form surface-attached communities, known as biofilms, are two factors contributing to the current antimicrobial resistance crisis.^{1,2} Adding to the crisis is an alarmingly reduced production of new antimicrobial agents. Additionally, many infections might be relapsing because of the inherent difficulty in eliminating biofilms using conventional antibiotic and disinfection treatments.^{3,4} Consequently, the number of health care—associated infections linked to antimicrobial resistant bacteria has increased at an alarming rate.^{2,5}

In 2016, economist Jim O'Neill estimated that the cumulative economic output at risk from antimicrobial resistance might exceed $100 trillion by 2050.⁶ Currently, treatment costs in Canada are nearly $1 billion each year, and likely exceed $20 billion in the US.^{2,7} Antimicrobial resistant bacteria are not limited to hospitals. For example, they are ubiquitous in nursing homes, food processing plants, and animal breeding facilities.^{1,8-11} There is thus a need to create new approaches to keep pathogenic microorganisms at bay. An ideal method will see the eradication of microbes without risk for developing resistance. Due to a mode of action markedly different from typical antibiotic drugs, photodynamic therapy (PDT) has been suggested for bacterial inactivation.^{12,13} PDT involves the use of a photosensitizer that, upon activation with light, generates toxic reactive oxygen species, such as singlet oxygen.^{14,15} Singlet oxygen is an “energized form” of molecular oxygen and is toxic to bacteria.^{13,16,17} This toxicity is due to singlet oxygen's high reactivity towards biomolecules including proteins, DNA and lipids.¹⁸ Certainly, due to their direct contact with patients, medical devices are recognized as a prominent nidus for bacterial infection.¹⁹ However, bacteria can survive and remain on dry inanimate surfaces, even after thorough cleaning and disinfection using bleach.²⁰⁻²²

It is well known in the prior art to employ photoactive dyes as photosensitizers. For example, dyes such as methylene blue, azure A, azure B, toluidine blue and new methylene blue have all been investigated for their direct use in photodynamic inactivation.⁵¹⁻⁵³ Moreover, some photosensitizer conjugates are known in the prior art, for example for use as antimicrobial textiles.⁵⁴

The need has arisen for new compositions useful as antimicrobials which utilize photosensitizers for PDT.

### SUMMARY

The present application is directed to photoactivated antimicrobial nanoparticles that can kill bacteria without releasing biocides. By coupling a photosensitizer to the surface of cellulose nanocrystals (CNCs), the inventors have obtained a concentrate that can be supplemented to aqueous and alcohol solutions, polymer blends, hydrogels and other materials to form photobioactive formulations. The antimicrobial nanomaterials or formulations containing the nanomaterials are then activated by otherwise harmless ambient light to kill microbes in contact with them.

The foregoing examples of the related art and limitations related thereto are intended to be illustrative and not exclusive. Other limitations of the related art will become apparent to those of skill in the art upon a reading of the specification and a study of the drawings.

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a composition comprising cellulose nanocrystals (CNCs) and photosensitizer molecules conjugated to the CNCs, wherein the composition generates reactive oxygen when exposed to light. In some embodiments the reactive oxygen is singlet oxygen. In some embodiments the photosensitizer molecules are adsorbed to the CNCs. In some embodiments the concentration of photosensitizer molecules in the composition is within the range of 0.03 to 0.075 mmol/100 mg. In some embodiments the photosensitizer molecules have a substantially planar conformation. In some embodiments the photosensitizer molecules may be selected from the group consisting of azure A, azure B, toluidine blue O (also referred to as toluidine blue), thionine acetate and cresyl violet. In one particular embodiment the photosensitizer molecules comprise azure A.

Another aspect of the invention relates to the use of a conjugate comprising cellulose nanocrystals (CNCs) and photosensitizer molecules comprising azure A as a photobiocidal disinfectant, wherein the photosensitizer molecules are adsorbed to the CNCs.

Another aspect of the invention relates to a method of disinfecting a surface comprising applying an aqueous solution comprising a composition comprising cellulose nanocrystals (CNCs) and photosensitizer molecules conjugated to the CNCs; and activating the photosensitizer molecules by applying light to the surface, thereby causing the composition to generate reactive singlet oxygen. In some embodiments the singlet oxygen is toxic to a broad spectrum of bacteria selected from the group consisting of gram-positive and gram-negative bacteria. In some particular embodiments the singlet oxygen is toxic to gram-negative bacteria selected from the group consisting of P. aeruginosa and K. pneumoniae. In some embodiments the composition, when light-activated, is significantly more toxic to the bacteria than light-activated photosensitizer molecules in a non-conjugated free form. In some particular embodiments the concentration of the composition in the solution is within the range of 1 ppm to 100 ppm. In some particular embodiments, the surface is a wound, such as a skin wound, a surface wound or an open wound.

Another aspect of the invention relates to a method of preparing a composition comprising cellulose nanocrystals (CNCs) and photosensitizer molecules conjugated to the CNCs, wherein the composition generates reactive oxygen when exposed to light, the method comprising providing a suspension of oxidized CNCs having an acidic pH; dispersing the photosensitizer molecules in the suspension; modifying the pH of the suspension to an alkaline pH to cause the photosensitizer molecules to conjugate to the CNCs to form the composition; and acidifying the suspension to yield a stable form of the composition. In some embodiments the alkaline pH is within the range of approximately 10-11. In some embodiments the suspension is maintained at the alkaline pH for at least 16 hours prior to acidifying the suspension. In some embodiments the oxidation level of the CNCs exceeds 0.75 mmol of CO₂H/gram. In some embodiments the acidifying comprises adding HCl to the suspension to adjust the pH to approximately 1.

Another aspect of the invention relates to a photobiocidal disinfectant formulation comprising a composition comprising cellulose nanocrystals (CNCs) and photosensitizer molecules conjugated to the CNCs, wherein the composition generates reactive oxygen when exposed to light. The formulation may comprise alcohol or an aqueous-compatible media. In some embodiments the media is a film-forming polymer or a hydrogel. In some particular embodiments the media is latex or acrylic paint having the composition dispersed therein. In particular embodiments, the aqueous-compatible media is a biocompatible hydrogel, and the concentration of the composition in the formulation is 0.01 to 10% by wt/v. In one embodiment, the photobiocidal disinfectant formulation comprising the biocompatible hydrogel is used for treating a wound.

Another aspect of the invention relates to medical devices comprising a photobiocidal disinfectant formulation comprising a composition comprising cellulose nanocrystals (CNCs) and photosensitizer molecules conjugated to the CNCs, wherein the composition generates reactive oxygen when exposed to light; and a biocompatible hydrogel.

Another aspect of the invention relates to a method of preparing a conjugate useful as biocidal disinfectant comprising functionalizing the surface of CNCs; oxidizing functional groups on the surface of the CNCs to provide a suspension of oxidized CNCs having an acidic pH; dispersing a photosensitizer in the suspension; modifying the pH of the suspension to an alkaline pH to cause the photosensitizer to adsorb to the CNCs to form the conjugate; and acidifying the suspension to yield a stable form of the conjugate. In some particular embodiments the photosensitizer is azure A. In some particular embodiments the functionalizing comprises carboxylation and the concentration of photosensitizer adsorbed to the surface of the CNCs exceeds 0.01 mmol/100 mg of CNC.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1A is a schematic representation of a photobiocidal composition comprising cellulose nanocrystals providing a template for anchoring photosensitizer molecules (shown as spheres).

FIG. 1B shows the chemical structure of an exemplary photosensitizer, the dye azure A.

FIG. 1C is a minimized geometry representation of azure A.

FIG. 1D is a flowchart summarizing a pH-mediated protocol for synthesizing a CNC-photosensitizer conjugate.

FIG. 1E is an energy diagram of CNC-AA chromophore/photosensitizer.

FIG. 2 is a graph showing azure A (AA) loading as a function of the level of CNC oxidation.

FIG. 3A is a graph showing the absorption spectrum of CNC-AA in an aqueous solution compared to free azure A dye (AA) in aqueous solution, showing that conjugation of AA to oxidized CNCs changes its absorption spectrum.

FIG. 3B is a graph showing the absorption spectrum of CNC-AA in an ethanol compared to free azure A dye (AA) in ethanol, showing that conjugation of AA to oxidized CNCs changes its absorption spectrum.

FIG. 4A is a graph showing the absorption spectrum of CNC-AA in an aqueous solution compared to free azure A dye (AA) in aqueous solution and to that of oxidized CNC with free azure A added, showing that conjugation of AA to oxidized CNCs, via a pH mediated protocol, changes its absorption spectrum that is different to mixing oxidized CNC with free azure A.

FIG. 4B is a graph showing the absorption spectrum of CNC-AA in phosphate-buffered saline (PBS), CNC-AA in reverse osmosis (RO) water and free azure A in PBS and RO water.

FIG. 5 is a graph showing the transient spectrum obtained 1 microsecond (μs) after laser pulsed excitation at 532 nm under an inert atmosphere (N2) of aqueous solutions of the free dye azure A (AA) and CNC-AA.

FIG. 6 is a graph showing time-resolved singlet oxygen emission signals at 1270 nm and the corresponding fitting to equation 1 for a solution of CNC-AA in D2O.

FIG. 7A is a bar graph showing biocidal activity against E. coli (ATCC25922) for samples kept for 20 minutes in the dark or under ambient light (LED) and containing either free dye azure A (AA), CNC alone, CNC supplemented with the free dye (CNC+AA) or the composition CNC-AA. The star symbol (*) indicates that the samples are significantly different (p<0.05). The concentration of the composition CNC-AA is 8 mg in 1 L. All the other samples have concentration adjusted to match the absorption of the composition.

FIG. 7B is a graph showing dose dependent effect against E. coli (ATCC25922) after 20 minutes of irradiation under ambient light (LED).

FIG. 8A is a graph showing time dependent photobiocidal effect of either free dye azure A (AA), CNC alone, CNC supplemented with the free dye (CNC+AA) or the composition CNC-AA against P. aeruginosa (PAO1) at a concentration of 8 mg/L.

FIG. 8B is a graph showing dose dependent photobiocidal effect of either free dye azure A (AA), CNC alone, CNC supplemented with the free dye (CNC+AA) or the composition CNC-AA against K. pneumoniae at a concentration of 8 mg/L.

FIG. 8C is a graph showing dose dependent photobiocidal effect of the composition CNC-AA against P. aeruginosa (PAO1) biofilms after 30 minutes of irradiation under ambient light (LED).

FIG. 8D is a graph showing dose dependent photobiocidal effect of the composition CNC-AA against P. aeruginosa (ATCC 15442) biofilms after 30 minutes of irradiation under ambient light (LED).

FIG. 9 is a bar graph showing biocidal activity against Staphylococcus aureus (ATCC29213) for samples kept for 20 minutes in the dark or under ambient light (LED) and containing either free dye azure A (AA), 8 mg/L CNC alone, 8 mg/L CNC supplemented with the free dye (CNC+AA) or the composition CNC-AA (8 mg/L). The star symbol (*) indicates that the samples are significantly different (p<0.05).

FIG. 10 is a bar graph showing the photobiocidal activity of various concentrations of test samples against Pseudomonas aeruginosa bacteria (PAO1). The test samples consisted of a CNC-AA sample where the dye azure A was coupled to the oxidized CNC via the applicant's pH mediated protocol (CNC-AA pH mediated); a sample where the coupling between CNC and the dye azure A was not performed via the pH mediated protocol (CNC-AA not pH mediated); a sample where the dye methylene blue (MB) was coupled to CNC via the applicant's pH mediated protocol (CNC-MB pH mediated); a sample of the free dye azure A which has undergone pH meditated change without CNC (AA pH); a sample of the free dye methylene blue (MB), and an untreated sample. Different concentrations of these samples (6.25 ppm=unfilled; 12 ppm=checkered, 25 ppm=shaded; and 50 ppm=hatched) were irradiated for 10 minutes under ambient light, using an LED set-up.

FIG. 11 is a photograph showing an acrylic paint sample comprising CNC-AA homogenously dispersed in the paint.

FIG. 12A is a graph showing the absorption spectrum of the singlet oxygen sensor ABDA in the presence of a painted surface irradiated with ambient light.

FIG. 12B is a graph showing evaluation of the singlet oxygen amount produced by the paint of FIG. 12A by following the peak absorbing at 401 nm over time of light exposure.

FIG. 13A is a graph showing the absorption spectrum of 18 ppm CNC-AA in a 0.017 M NaDC hydrogel. An initial absorption spectrum and a spectrum after exposure to ambient light for 5 hours is shown.

FIG. 13B is a graph showing the absorption spectrum of 18 ppm CNC-AA in a 0.034 M NaDC hydrogel. An initial absorption spectrum and a spectrum after exposure to ambient light for 5 hours is shown.

FIG. 13C is a graph showing the absorption spectrum of 18 ppm CNC-AA in a 0.051 M NaDC hydrogel. An initial absorption spectrum and a spectrum after exposure to ambient light for 5 hours is shown.

FIG. 13D is a graph showing the absorption spectrum of 36 ppm CNC-AA in a 0.017 M NaDC hydrogel. An initial absorption spectrum and a spectrum after exposure to ambient light for 5 hours is shown.

FIG. 13E is a graph showing the absorption spectrum of 36 ppm CNC-AA in a 0.034 M NaDC hydrogel. An initial absorption spectrum and a spectrum after exposure to ambient light for 5 hours is shown.

FIG. 13F is a graph showing the absorption spectrum of 36 ppm CNC-AA in a 0.051 M NaDC hydrogel. An initial absorption spectrum and a spectrum after exposure to ambient light for 5 hours is shown.

FIG. 13G is a graph showing the absorption spectrum of 54 ppm CNC-AA in a 0.017 M NaDC hydrogel. An initial absorption spectrum and a spectrum after exposure to ambient light for 5 hours is shown.

FIG. 13H is a graph showing the absorption spectrum of 54 ppm CNC-AA in a 0.034 M NaDC hydrogel. An initial absorption spectrum and a spectrum after exposure to ambient light for 5 hours is shown.

FIG. 13I is a graph showing the absorption spectrum of 54 ppm CNC-AA in a 0.051 M NaDC hydrogel. An initial absorption spectrum and a spectrum after exposure to ambient light for 5 hours is shown.

FIG. 14 is a graph showing singlet oxygen detection using the singlet oxygen probe ABDA after exposure of a sodium deoxycholate hydrogel (36 ppm CNC-AA, 0.017 M NaDC) to ambient light. The absorption of the probe ABDA between 300 and 400 nm decreases significantly with time, indicating the production of singlet oxygen.

FIG. 15 is a photograph showing CNC-AA homogenously dispersed in gelatine hydrogel.

FIG. 16A shows the chemical structure of the dye safranin 0 (SO).

FIG. 16B is a minimized geometry representation of safranin 0.

FIG. 16C is a graph showing the absorption spectrum of CNC-SO in PBS, CNC-SO in reverse osmosis water (RO) and free safranin 0 in PBS and reverse osmosis water.

FIG. 16D is a bar graph comparing the antimicrobial activity of CNC-SO and free SO in PBS solution after exposure to light for 20 minutes. The CNC-SO concentration used was 100 ppm. The concentration of SO was adjusted to match the absorption value of the CNC-SO.

FIG. 17A shows the chemical structure of the dye methylene violet 3RAX (MLV);

FIG. 17B is a minimized geometry representation of methylene violet 3RAX.

FIG. 17C is a graph showing the absorption spectrum of CNC-MLV in PBS, CNC-MLV in reverse osmosis water and free MLV in PBS and reverse osmosis water.

FIG. 17D is a bar graph comparing the antimicrobial activity of CNC-MLV and free MLV in PBS solution after exposure of light for 20 minutes. The CNC-MLV concentration used was 100 ppm. The concentration of free MLV was adjusted to match the absorption value of the CNC-MLV.

FIG. 18A shows the chemical structure of the dye azure B (AB).

FIG. 18B is a minimized geometry representation of azure B.

FIG. 18C is a graph showing the absorption spectrum of CNC-AB in PBS, CNC-AB in reverse osmosis water and free AB in PBS and reverse osmosis water;

FIG. 18D is a bar graph comparing the antimicrobial activity of CNC-AB and free AB in PBS solution after exposure to light for 15 minutes. The CNC-AB concentration used was 100 ppm. The concentration of free AB was adjusted to match the absorption value of the CNC-AB.

FIG. 19A shows the chemical structure of the dye toluidine blue (TB).

FIG. 19B is a minimized geometry representation of toluidine blue.

FIG. 19C is a graph showing the absorption spectrum of CNC-TB in PBS, CNC-TB in reverse osmosis water and free TB in PBS and reverse osmosis water;

FIG. 19D is a bar graph comparing the antimicrobial activity of CNC-TB and free TB in PBS solution after exposure to light for 10 minutes. The CNC-TB concentration used was 6.25 ppm. The concentration of free TB was adjusted to match the absorption value of the CNC-AB.

FIG. 20A shows the chemical structure of the dye thionine acetate (TA).

FIG. 20B is a minimized geometry representation of thionine acetate (TA).

FIG. 20C is a graph showing the absorption spectrum of CNC-TA in PBS, CNC-TA in reverse osmosis water and free TA in PBS and reverse osmosis water;

FIG. 20D is a bar graph comparing the antimicrobial activity of CNC-TA and free TA in PBS solution after exposure to light for 10 minutes. The CNC-TA concentration used was 6.25 ppm. The concentration of free TA was adjusted to match the absorption value of the CNC-TA.

FIG. 21A shows the chemical structure of the dye rhodamine 6G (R6G).

FIG. 21B is a minimized geometry representation of rhodamine 6G.

FIG. 21C is a graph showing the absorption spectrum of CNC-R6G in PBS, CNC-R6G in reverse osmosis water and free R6G in PBS and reverse osmosis water.

FIG. 21D is a bar graph comparing the antimicrobial activity of CNC-R6G and free R6G in PBS solution after exposure to light for 15 minutes. The CNC-R6G concentration used was 100 ppm. The concentration of free R6G was also 100 ppm.

FIG. 22A shows the chemical structure of the dye cresyl violet (CV).

FIG. 22B is a minimized geometry representation of cresyl violet.

FIG. 22C is a graph showing the absorption spectrum of CNC-CV in PBS, CNC-CV in reverse osmosis water and free CV in PBS and reverse osmosis water.

FIG. 23A shows the chemical structure of the dye acriflavine (AF).

FIG. 23B is a minimized geometry representation of acriflavine (AF).

FIG. 23C is a graph showing the absorption spectrum of CNC-AF in PBS, CNC-AF in reverse osmosis water and free AF in PBS and reverse osmosis water.

FIG. 24 is a photograph showing CNC-AA and CNC-TB homogenously dispersed in hydrogels sample containing: (A) hydroxyethyl cellulose and propylene glycol in reverse osmosis water; (B) hydroxyethyl cellulose and propylene glycol in reverse osmosis water; (C) hydroxyethyl cellulose, carboxymethyl cellulose and propylene glycol in reverse osmosis water; (D) carboxymethyl cellulose and propylene glycol in reverse osmosis water; (E) sodium polyacrylate type I in reverse osmosis water; (F) sodium polyacrylate type II in reverse osmosis water.

FIG. 25A is a graph showing the absorption spectrum of AA (0.01%) in reverse osmosis water and CNC-AA (0.01%) in hydrogels sample containing: (A) hydroxyethyl cellulose and propylene glycol in reverse osmosis water; (B) hydroxyethyl cellulose and propylene glycol in reverse osmosis water; (C) hydroxyethyl cellulose, carboxymethyl cellulose and propylene glycol in reverse osmosis water; (D) carboxymethyl cellulose and propylene glycol in reverse osmosis water; (E) sodium polyacrylate type I in reverse osmosis water; (F) sodium polyacrylate type II in reverse osmosis water.

FIG. 25B is a graph showing the absorption spectrum of TB (0.01%) in reverse osmosis water CNC-TB (0.01%) in hydrogels sample containing: (A) hydroxyethyl cellulose and propylene glycol in reverse osmosis water; (B) hydroxyethyl cellulose and propylene glycol in reverse osmosis water; (C) hydroxyethyl cellulose, carboxymethyl cellulose and propylene glycol in reverse osmosis water; (D) carboxymethyl cellulose and propylene glycol in reverse osmosis water; (E) sodium polyacrylate type I in reverse osmosis water; (F) sodium polyacrylate type II in reverse osmosis water.

FIG. 26 is a graph showing the singlet oxygen luminescence signal at 1270 nm for CNC-AA (0.01%) in hydrogels sample containing: (A) hydroxyethyl cellulose and propylene glycol in reverse osmosis water; (B) hydroxyethyl cellulose and propylene glycol in reverse osmosis water; (C) hydroxyethyl cellulose, carboxymethyl cellulose and propylene glycol in reverse osmosis water; (D) carboxymethyl cellulose and propylene glycol in reverse osmosis water.

FIG. 27A is a graph showing the antimicrobial activity upon light activation with LED for 20 minutes against E. coli of CNC-AA (0.01%) in hydrogels sample containing: (A) hydroxyethyl cellulose and propylene glycol in reverse osmosis water; (B) hydroxyethyl cellulose and propylene glycol in reverse osmosis water; (C) hydroxyethyl cellulose, carboxymethyl cellulose and propylene glycol in reverse osmosis water; (D) carboxymethyl cellulose and propylene glycol in reverse osmosis water; (E) sodium polyacrylate type I in reverse osmosis water; (F) sodium polyacrylate type II in reverse osmosis water. The data were obtained via the direct gel method.

FIG. 27B is a graph showing the antimicrobial activity upon light activation with LED for 20 minutes against S. aureus of CNC-AA (0.01%) in hydrogels sample containing: (A) hydroxyethyl cellulose and propylene glycol in reverse osmosis water; (B) hydroxyethyl cellulose and propylene glycol in reverse osmosis water; (C) hydroxyethyl cellulose, carboxymethyl cellulose and propylene glycol in reverse osmosis water; (D) carboxymethyl cellulose and propylene glycol in reverse osmosis water; (E) sodium polyacrylate type I in reverse osmosis water; (F) sodium polyacrylate type II in reverse osmosis water. The data were obtained via the direct gel method.

FIG. 27C is a graph showing the antimicrobial activity upon light activation with LED for 20 minutes against E. coli of CNC-TB (0.01%) in hydrogels sample containing: (A) hydroxyethyl cellulose and propylene glycol in reverse osmosis water; (B) hydroxyethyl cellulose and propylene glycol in reverse osmosis water; (C) hydroxyethyl cellulose, carboxymethyl cellulose and propylene glycol in reverse osmosis water; (D) carboxymethyl cellulose and propylene glycol in reverse osmosis water; (E) sodium polyacrylate type I in reverse osmosis water; (F) sodium polyacrylate type II in reverse osmosis water. The data were obtained via the direct gel method.

FIG. 27D is a graph showing the antimicrobial activity upon light activation with LED for 20 minutes against S. aureus of CNC-TB (0.01%) in hydrogels sample containing: (A) hydroxyethyl cellulose and propylene glycol in reverse osmosis water; (B) hydroxyethyl cellulose and propylene glycol in reverse osmosis water; (C) hydroxyethyl cellulose, carboxymethyl cellulose and propylene glycol in reverse osmosis water; (D) carboxymethyl cellulose and propylene glycol in reverse osmosis water; (E) sodium polyacrylate type I in reverse osmosis water; (F) sodium polyacrylate type II in reverse osmosis water. The data were obtained via the direct gel method.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

This application relates to compositions comprising cellulose nanocrystals (CNCs) and photosensitizer molecules conjugated to the CNCs. In some embodiments the compositions generate reactive oxygen when exposed to light. Methods of preparing the compositions and using the compositions or formulations containing the compositions as biocidal disinfectants are also described.

This application also relates to formulations and medical devices comprising cellulose nanocrystals (CNCs) and photosensitizer molecules conjugated to the CNCs for wound disinfection. In some embodiments, the medical devices are bandages, wound dressings, pads, gauzes, sponges, foams, or calcium alginate formulations containing the CNC and photosensitizer conjugates. In one embodiment, the medical devices further contain pharmaceutical compositions, such as antibiotics.

CNCs are a biomaterial that can be extracted from wood fibre. As described in Leng et al., 2017,⁵⁵ CNCs are rod-shaped crystals that have a high crystalline content, high mechanical strength, and many other desirable properties. CNCs have a very large surface area at the nano-scale making them an ideal template for housing other molecules.⁵⁶

Photosensitizers have been investigated for their use in photodynamic inactivation. As discussed above, non-conjugated photosensitizers have been proposed for photodynamic inactivation. A number of dye-labelled CNCs have also been prepared and used for different purposes.⁵⁵ The present invention is directed to engineered compositions for enhancing the biocidal efficacy of photosensitizers by coupling the photosensitizers to CNCs. In some embodiments the photosensitizers are coupled to the CNCs by a pH mediated protocol.

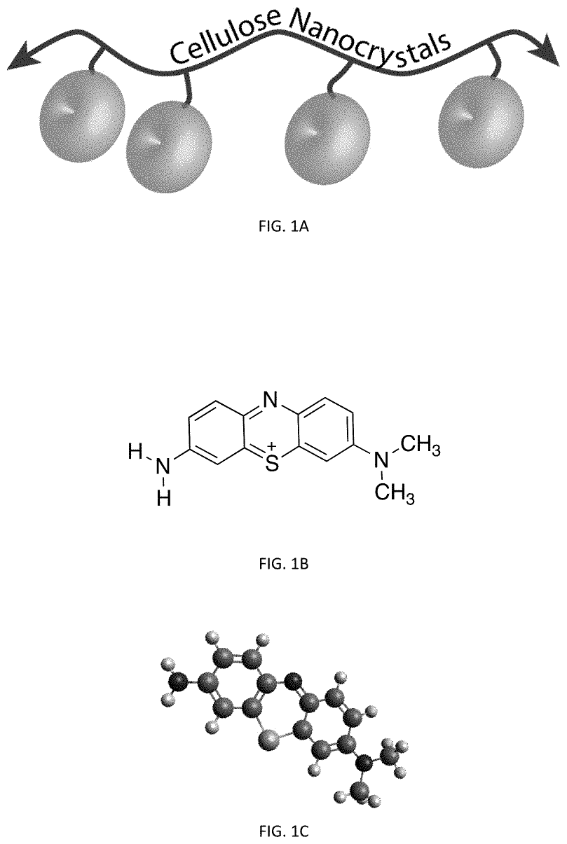

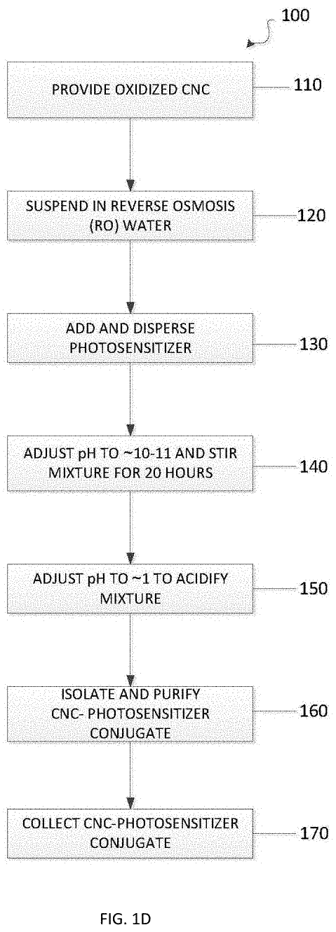

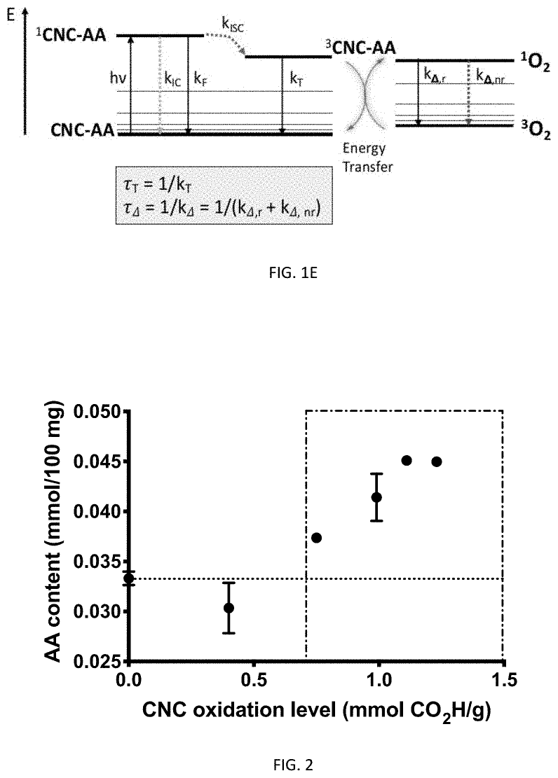

FIG. 1A is a schematic representation of a composition useful as a photobiocidal agent wherein cellulose nanocrystals provide a template for anchoring photosensitizer molecules. In some embodiments the photosensitizer may be a cationic dye with acidic protons. For example, phenothiazine dyes can be employed as photosensitizers. The photosensitizer molecules are fixed to the CNCs, for example by adsorption, so that the CNC-photosensitizer composition (conjugate) is stable, for example in aqueous solutions.

In one embodiment, the phenothiazine dye azure A (FIGS. 1B and 1C) was investigated as a photosensitizer. Azure A is a derivative of methylene blue. The latter is a known photosensitizer that has potent bactericidal activity and presents low toxicity to humans.²⁷⁻³² Furthermore, methylene blue is used clinically for decontamination of freshly frozen plasma units by the Swiss and German Red Cross²⁹ and has received regulatory approval for photodynamic therapy of dental infectious diseases.^{33,34}

### Preparation of CNC-Photosensitizer Conjugates

In order to prepare dye modified CNC compositions (conjugates), for example comprising azure A, the inventors have developed a pH mediated protocol. The conjugate comprising azure A is referred to herein as CNC-AA. Other conjugates prepared by the same or substantially the same protocol are described below. In the case of preparation of CNC-AA, the protocol advantageously utilizes a pH dependent equilibrium between azure A and its neutral, free base form. The inventors determined that using oxidized CNC maximized the desired photobiocidal properties of the conjugates. By first dispersing azure A in a suspension of oxidized CNC, kept at acidic pH, and then modifying the pH to ˜10-11 the inventors produced a neutral, free base form of azure A. This makes azure A much less soluble in water and forces its conjugation to the CNC substrate/template.

One embodiment of the pH mediated protocol of the present invention is summarized in FIG. 1D. In accordance with synthetic method 100, a supply of oxidized CNC was provided in step 110. More particularly, oxidized CNC was prepared via well-established TEMPO mediated oxidation (TEMPO=(2,2,6,6-tetramethylpiperidin-1-yl)oxyl)) with commercially available bleach solutions.^{23,24} The oxidized CNC (575 mg, 1.23 mmol CO₂H/g, 0.741 mmol CO₂H) was suspended in 60 mL of reverse osmosis (RO) water (step 120). Added to this was azure A chloride (216 mg, 0.741 mmol) and once the azure A photosensitizer was dispersed (step 130), the pH was adjusted to ˜10-11 (step 140). This mixture was left to stir for 20 hours. After 20 hours, the mixture was acidified with 6 M HCl and the pH adjusted to ˜1 (step 150). The CNC-AA reaction product was then isolated and purified (step 160). In this example, 4 g of NaCl was added and the mixture was collected into centrifuge tubes for centrifugation (4700×g, 10 min) and isolation. The resulting pellet was suspended in water and centrifuged again (4700× g, 10 min). This latter procedure was then carried out in 0.05 M HCl (3×), RO water (3×) and ethanol (5×). Finally, the resulting pellet was transferred to a thimble for Soxhlet purification in ethanol (carried out for 20 hours). After Soxhlet purification, the dark blue solid was collected and the ethanol was evaporated under reduced pressure until constant weight (424 mg) was obtained (step 170).

The pH mediated coupling protocol described above is believed to be a novel synthetic method that significantly reduces the cost of CNC-AA production and ensures its scalability. Some important features of the protocol include: 1) a higher level of CNC oxidation is better for dye fixation, 2) alkaline mediated attachment is better for dye fixation and 3) cationic dyes with acidic protons, such as azure A, work better for dye fixation. As will be appreciated by a person skilled in the art, the specific steps of the pH mediated protocol can vary in different embodiments of the invention, for example depending upon the photosensitizer used, concentration of reagents, temperature, pressure, or other reaction parameters. For example, after adjusting the mixture to an alkaline pH (step 140), the mixture could be stirred for 16 hours or more rather than 20 hours. In some embodiments, the mixture is stirred for 10 hours or more, 12 hours or more, 14 hours or more, 16 hours or more, or 18 hours or more. In one embodiment, the mixture is stirred for 16 hours or more.

FIG. 2 shows the relationship between the CNC level of oxidation and the amount of dye coupled to CNC via the inventors' pH mediated synthesis. The level of oxidation is quantified by titrating CNC with NaOH using a pH probe. While this process is time consuming, it is the most accurate methodology to estimate the number of carboxylic acid groups present per gram of material. The amount of dye coupled to the CNC surface (dye loading) is evaluated by elemental analysis. Since in this example azure A is the only species with nitrogen (FIG. 1B), the amount of dye per gram of material can thus be back calculated. The inventors' data indicates that, in this example, the CNC oxidation level is preferably at least 0.75 mmol of CO₂H/gram of material in order to successfully couple the dye to the CNC (FIG. 2). In some embodiments, the CNC oxidation level is at least 0.25, at least 0.5 mmol of CO₂H/gram, at least 0.75 mmol of CO₂H/gram, or at least 1 mmol of CO₂H/gram. In one embodiment, the CNC oxidation level is at least 0.75 mmol of CO₂H/gram. In some embodiments, the oxidation level of the CNCs is within the range of 0.5-3 mmol of CO₂H/gram, 0.75-2 mmol of CO₂H/gram, or 1-1.5 mmol of CO₂H/gram. In one embodiment, the oxidation level of the CNCs is within the range of 1-1.5 mmol of CO₂H/gram.

Other means for preparing CNC-photosensitizer conjugates may be envisaged by a person skilled in the art. In the inventors' coupling protocol described above the surface of the CNCs is functionalized by oxidization of available hydroxyl groups on the cellulose to yield carboxyl (COO—) groups. In alternative embodiments, the surface of the CNCs may be functionalized by other means to facilitate conjugation of AA or other photosensitizers. For example, the surface of the cellulose may be modified with other functional groups such as sulfate groups (OSO3-), aldehyde groups (CHO), amino groups (NH2) or thiol groups (SH). The functional groups can then subsequently be utilized for the addition of desired compounds to form a conjugate. Other means of CNC surface modification which may enhance adsorption of photosensitizers include esterificiation, silylation and polymer grafting.

### Photophysical Characterization of CNC-AA

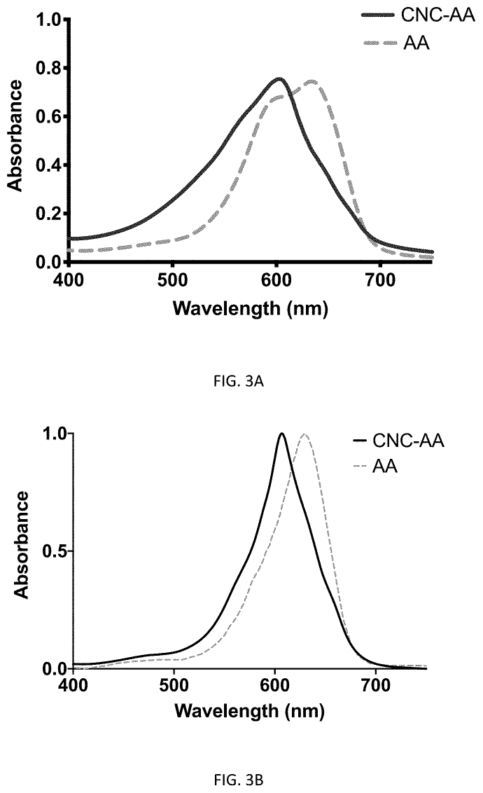

The photophysical properties of the CNC-AA conjugate which make it useful as a photoantimicrobial agent were investigated by the inventors. Since CNC-AA is activated by light, the inventors investigated the absorption profile of CNC-AA in aqueous solution. As shown in FIG. 3A, CNC-AA presents an absorption profile significantly different compared to the free dye in solution. Indeed, the absorption maximum of CNC-AA (solid trace, FIG. 3A) is shifted towards lower wavelengths compared to the absorption spectrum of free azure A (dashed trace, FIG. 3A). Furthermore, CNC-AA reveals an absorption profile with a single peak and a weak shoulder, while azure A has two characteristic peaks associated to a monomer form at 650 nm and a dimer species around 600 nm (FIG. 3A).^{35,36} The absorption spectrum of CNC-AA is also broader than the one of azure A. All these characteristics are responsible for the difference in color observed for the aqueous solutions. Indeed, azure A absorbing mainly in the red region of the visible spectrum (FIG. 3A) gives a blue color to the solution. On the other hand, an aqueous solution of CNC-AA is purple as its absorption profile stretches mainly over the yellow region of the visible spectrum.

In addition, the inventors investigated the absorption profile of CNC-AA in alcohol solution. As shown in FIG. 3B, CNC-AA presents an absorption profile significantly different compared to the free dye in solution. Similarly, to what was observed in aqueous solution, the absorption maximum of CNC-AA (solid trace, FIG. 3B) is shifted towards lower wavelengths compared to the absorption spectrum of free azure A (dashed trace, FIG. 3B). Indeed, CNC-AA reveals an absorption profile in ethanol with a single peak located at 604 nm, while azure A exhibits its maximum of absorption at 634 nm in ethanol (FIG. 3B). The absorption spectrum of CNC-AA is also broader than the one of azure A.

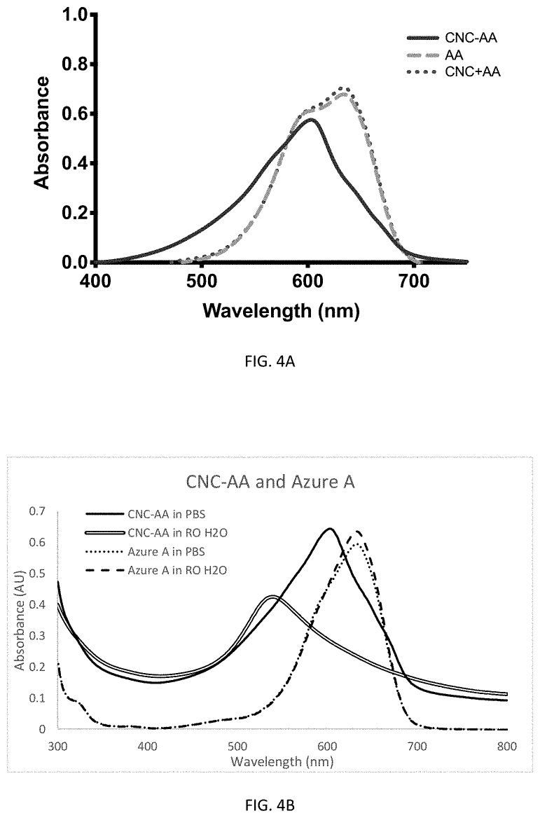

To address the possibility that the optical changes observed for CNC-AA are due to the adsorption of the dye on the surface of CNC, a control experiment was performed where the absorption spectrum of free azure A in the presence of oxidized CNC was recorded. As shown in FIG. 4A, the absorption profile of azure A presents only minimal changes when oxidized CNC is added to the solution. This result is important as it confirms that the optical changes observed for the CNC-AA are due to the coupling of the dye to the surface of CNC under the pH mediated protocol described above.

### Production of Reactive Oxygen by CNC-Photosensitizer Conjugates

With the exception of some anaerobic organisms, molecular oxygen (O₂) is vital for animals, plants, and bacteria. However, its necessity often conceals the fact that O₂ is a toxic and strongly oxidizing molecule.¹⁵ Interestingly, under physiological conditions, O₂ is fairly inert towards most organic and biological molecules, as it does not combine immediately with them.¹⁵ This begs the question, why is O₂ toxic if it is inert? It is now understood that O₂ toxicity is linked to its metabolism, allowing for its reduction and generation of a variety of reactive oxygen species.¹⁵ In other words, these reactive oxygen species are key in O₂ toxicity. The reactive oxygen species of particular importance to the present invention is singlet oxygen (¹O₂), which corresponds to the excited state of molecular oxygen (O₂). Due to its distinctive electronic structure with two paired electrons, singlet oxygen is a non-radical with unique chemistry.¹⁵ Singlet oxygen reacts readily with lipids, DNA and proteins, leading to formation of endoperoxides, peroxides, and other unstable intermediates, thus causing high toxicity.¹⁵

Photosensitization is the most common method to generate singlet oxygen as shown in the energy diagram (FIG. 1E). Photosensitization requires light and a specific chromophore, commonly referred to as a photosensitizer.¹⁵ In this example, the conjugate CNC-AA is the chromophore/photosensitizer. In the ground state CNC-AA absorbs a photon (hv) yielding an excited single state (¹CNC-AA). The latter can return to the ground state by emission of light (fluorescence, k_{F}) or by non-radiative decay (internal conversion, k_{IC}). The single state (¹CNC-AA) can undergo intersystem crossing (ISC) to the triplet state (³CNC-AA). Subsequently, ³CNC-AA can then either reduce O₂ to form the superoxide anion (O₂⁻, Type I mechanism) or transfer its energy to O₂, yielding singlet oxygen (¹O₂, Type II mechanism). Accordingly, with reference to the energy diagram shown in FIG. 1E, the triplet manifold ³CNC-AA can deactivate to CNC-AA (triplet rate constant, k_{T}). Furthermore, ³CNC-AA can transfer its energy to ground state O₂ (³O₂), thus generating ¹O₂. If left unreacted, ¹O₂ is converted back into inert oxygen (³O₂), by emission of a photon (radiative rate constant, (k_{Δ,r}) or by solvent deactivation via non-radiative pathway (k_{Δ,nr}). In the box legend of FIG. 1E, τT is the triplet lifetime and τ_{Δ} is the ¹O₂ lifetime.

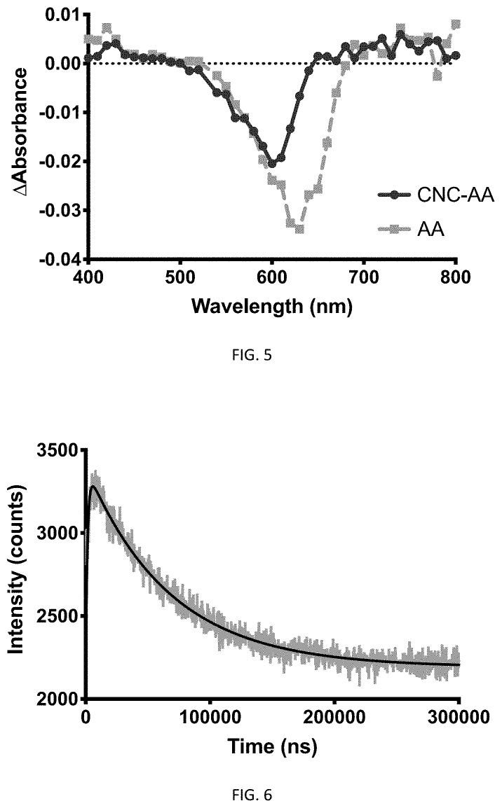

Since the first step in singlet oxygen generation via photosensitization is intersystem-crossing of the excited dye, the triplet state of the CNC-AA was characterized. First, the inventors identified the triplet state of the free dye in solution. As shown in FIG. 5, the transient spectrum of free azure A in aqueous solution under inert atmosphere (N2) presents three distinct areas: there is a positive absorbance below 550 nm, followed by a strong negative absorbance between 550 nm and 700 nm, which becomes positive again after that wavelength. This spectrum agrees with the literature.^{37,38} The negative region corresponds to the depopulation of the ground state, while the two positive regions are associated to the triplet-triplet absorption spectrum of azure A.^{37,38} When the same experiment is performed with CNC-AA, a very similar transient spectrum is obtained. The same shift towards lower wavelength, as observed in the steady state in FIG. 3A, exists also in the negative portion of the transient spectrum. Therefore, the Applicant's result is indicative of the ability of CNC-AA to intersystem cross to the triplet state. The inventors' data also shows an increase in the triplet state lifetime of CNC-AA compared to the free dye in solution. Indeed, the triplet state of azure A in solution lasts for about 10 μs, while the one of CNC-AA is extended to 18 μs. This increase in lifetime is important as it should lead to an increase in reactive oxygen species (ROS) production.

Since the CNC-AA is capable of intersystem-crossing to a triplet state, ³CNC-AA, the inventors investigated its ability to generate singlet oxygen. Singlet oxygen is emissive in the near infrared region of the electromagnetic spectrum with a maximum at 1270 nm.¹⁵ Thus, its production can be directly monitored. However, as only one singlet oxygen molecule in 100,000 is emissive in aqueous environment, its detection requires a very sensitive customized instrumentation.^{39,40} Briefly, a diode-pumped pulsed Nd:YAG laser working at 1 kHz repetition rate at 532 nm (12 mW, 1.2 ρJ per pulse) was used for excitation of the CNC-AA sample. The singlet oxygen emission exiting from the sample was then detected at 90° angle via an Hamamatsu NIR detector (peltier cooled at −62.8° C. operating at 800 V) coupled to a grating monochromator. Photon counting was then achieved with a multichannel scaler (NanoHarp 250, PicoQuant Gmbh, Germany).^{39,40}

The production of singlet oxygen is essentially a two-step process in which light energy is first absorbed by CNC-AA and then transferred to molecular oxygen to produce singlet oxygen (¹O₂). According to the energy diagram set forth above, the basic kinetic parameters that contribute to singlet oxygen production and decay are the ³CNC-AA lifetime, TT, and the singlet oxygen lifetime, τΔ. Therefore, the emission signal (St) of singlet oxygen detected at 1270 nm presents a rise and decay bi-exponential behaviour, which can be modelled by the following expression (equation 1).^{39,40}

$${S}_{t} = {S}_{0} \times \frac{{τ}_{Δ}}{{τ}_{Δ} - {τ}_{T}} \times ( {e}^{- t / {τ}_{Δ}} - {e}^{- t / {τ}_{T}} ) \qquad ( 1 )$$

The time-resolved near-infrared emission signals at 1270 nm for CNC-AA was collected in aqueous environment (D₂O) on the instrument described above. The signal is presented in FIG. 6, which could be fitted with equation 1, above. A singlet oxygen lifetime (τΔ) of 67 μs was obtained, which is in good agreement with singlet oxygen lifetime in this solvent.⁴¹

### Photobactericidal Activity of CNC-AA

The inventors assessed the photoantimicrobial activity of CNC-AA against various bacterial strains of hospital relevance for the desired disinfectant properties. In the experimental set-up, the viability of bacteria as a response to intensity of white light exposure and concentration of disinfectant was monitored. This was carried out by calculating the number of bacterial colonies present in solution before and after exposure.

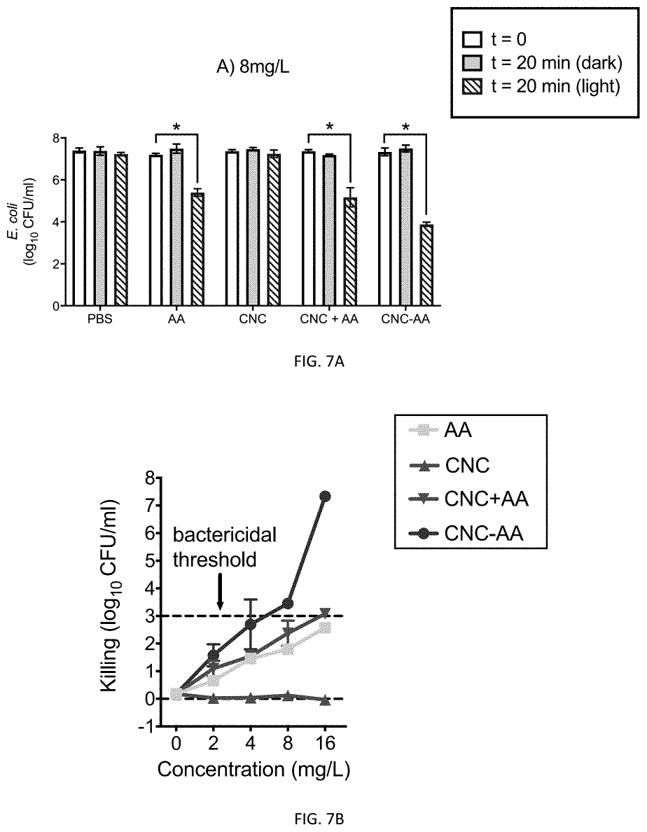

According to the experimental protocol, a suspension of cells (2.6×108 cells/ml ±0.5 log) was treated with either AA, CNC or CNC+AA or CNC-AA or PBS (untreated control) at different concentration (FIGS. 7-9). For each set of conditions, the initial culture was equally divided into two. One part was light treated while the other was kept in the dark. After different time periods (FIGS. 7-9), bactericidal activity was quantified by counting colony forming units (CFU) on agar plates. CFU count is a well-established method, in which each colony growing on plate results from the multiplication of one bacterial cell that was present in solution.⁴² Therefore, by counting the number of bacterial colonies, the inventors were able to calculate the number of cells present in the samples.⁴² Comparison of the number of CFUs in the treated samples (AA, CNC, CNC+AA and CNC−AA) to untreated (PBS) kept in the dark, enabled the inventors to quantify the dark bactericidal activity of the test agents. Comparison of the number of CFUs in the treated samples (AA, CNC, CNC+AA and CNC-AA) to untreated (PBS) placed under light, enabled the inventors to quantify the specific photo-bactericidal activity of the test agents. Killing effect was calculated using the following expression (equation 2).

Killing=Log 10(#CFU_{Untreated})−Log 10(CFU_{treated}) (2)

As shown in FIG. 7a, CNC-AA displayed bactericidal activity at a concentration as low as 8 mg in 1 liter of water after 20 minutes irradiation under white light. The antimicrobial effect of CNC-AA is due to light activation, as it presents no toxicity in the dark (FIG. 7A). Importantly, at the concentration used herein, CNC itself presents no toxicity against E. coli. Finally, the free dye AA or the free dye in presence of oxidized CNC (CNC+AA) show similar toxicity, which is significantly reduced compared to CNC-AA (FIG. 7A). It is important to note that the optical density of all the samples is comparable, suggesting that approximately the same amount of light is absorbed by all the samples. Therefore, coupling the photosensitizer AA to CNC (CNC-AA) provides a notable gain of function. FIG. 7B shows a dose-dependent effect of the antimicrobial agent tested upon 20 min of irradiation with white light. If the concentration of CNC-AA is increased to 16 mg/L, one can see that no E. coli could be detected, as almost 8 log 10 killing is obtained (FIG. 7B). The CNC-AA conjugate thus exceeds the minimum approved activity of common disinfectant, which is set at reduction of population by 3 log₁₀.

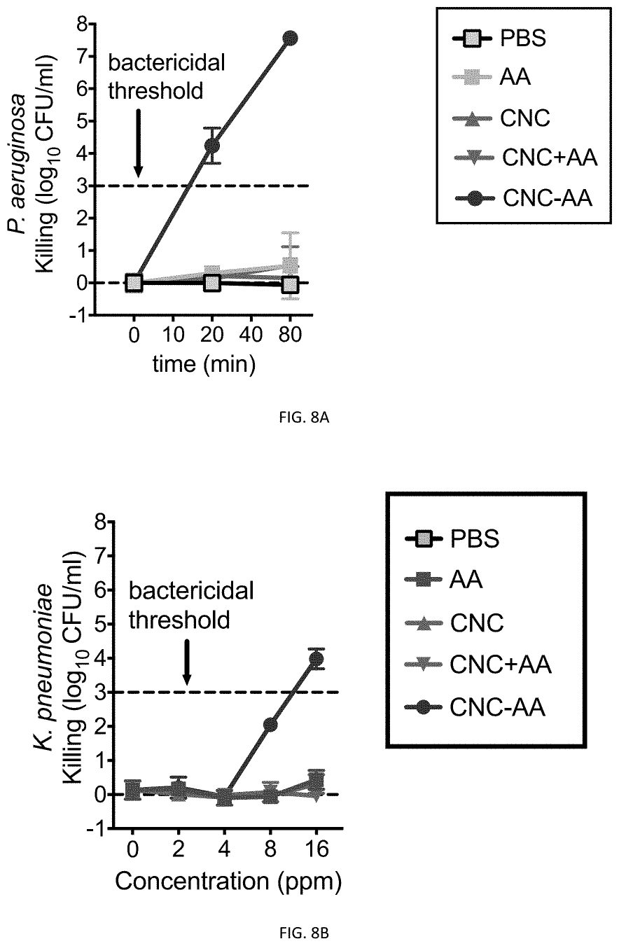

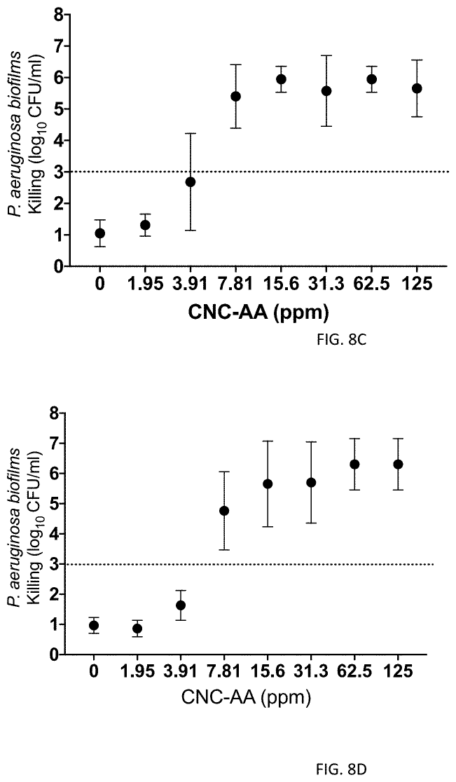

Experiments were also conducted using two other Gram-negative hospital pathogens, Pseudomonas aeruginosa and Klebsiella pneumoniae. These bacterial strains are known for their multidrug resistance.⁴³⁻⁴⁶ The presence of several copies of microbial efflux pumps in these strains has become broadly recognized as major components of microbial resistance, as these pumps expel a variety of structurally diverse compounds with differing modes of action.⁴⁷⁻⁴⁹ In fact, photosensitizers may have very little effect on some bacteria, such as P. aeruginosa, because photosensitizers are effectively expelled from the bacteria via multidrug efflux pumps.⁵⁰ As seen in FIGS. 8A-8B, CNC-AA is the only effective drug of the ones tested against these two strains. In addition, it meets the bacterial threshold of 3 log₁₀ kill for disinfectant at a concentration of 16 mg/L for 20 min of irradiation under ambient light. These results are outstanding, as they show an unexpected mode of action for CNC-AA.

Importantly, P. aeruginosa are also known for their capacity to form biofilms in many environments.^{57,58} Biofilms provide bacteria with an enormous advantage as they render antimicrobial treatment inefficient. In fact, biofilms are not only responsible for the majority of opportunistic bacterial infections in medicine and dentistry,^{3,4} they are also considered as the main contributor to the development and maintenance of chronic wounds.^{43,59} Especially, P. aeruginosa biofilms have been reported to delay wound healing in various type of chronic wounds, burns and surgical incisions.⁵⁹⁻⁶¹

The inventors evaluated the ability of CNC-AA to eradicate biofilms. The minimum biofilm eradication concentration of CNC-AA under ambient light exposure was determined using a MBEC™—high throughput assay.⁶² As shown in FIGS. 8C-8D, CNC-AA leads to complete eradication of bacterial biofilms at a concentration as low as 8 mg/L (8 ppm) after 30 minutes of ambient light exposure. These results are outstanding and quite unexpected as P. aeruginosa biofilms are known for their robustness as they both inhibit the diffusion of antimicrobial agent and deactivate potential harmful reactive oxygen species.^{63,64} Furthermore, it has been shown that significantly higher concentration (0.01% wt/wt or 10 g/L) combined with laser irradiation at specific wavelengths are necessary for free phenothiazine dyes, such as methylene blue, to eradicate P. aeruginosa biofilms.⁶⁵

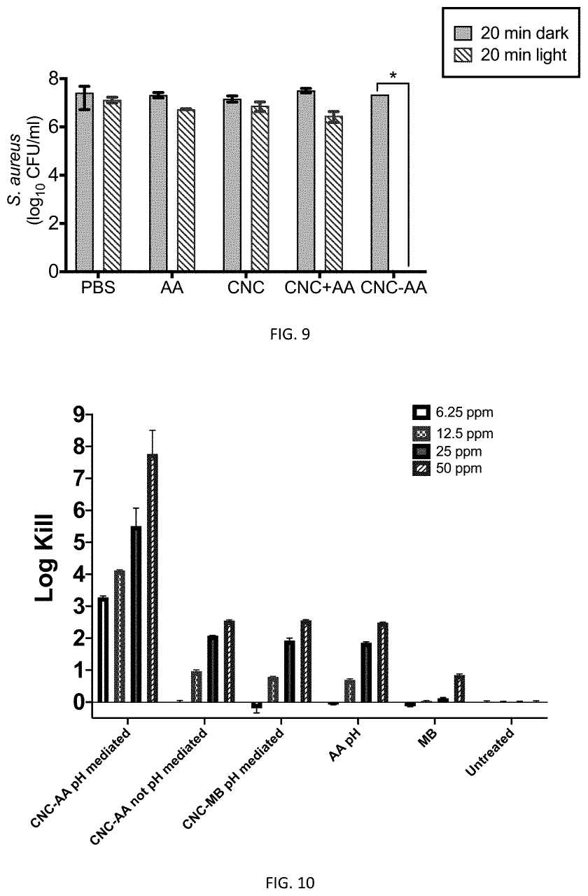

The efficacy of CNC-AA was also tested against Staphylococcus aureus, which is a Gram-positive strain. CNC-AA shows a very strong photo-biocidal effect at a concentration as low as 4 mg/L. This result demonstrates broad-spectrum antibacterial activity of CNC-AA (FIG. 9).

In further comparative experiments, CNC-AA produced by the pH mediated protocol described above and other test samples were tested for their photobiocidal activity against P. aeruginosa. With reference to FIGS. 9-10, the test samples consisted of a CNC-AA sample where the dye azure A was coupled to the oxidized CNC via the applicant's pH mediated protocol described above and summarized in FIG. 1D (CNC-AA pH mediated); a sample where the coupling between CNC and the dye azure A was not performed via the pH mediated protocol (the steps described above were followed except for step 140 of FIG. 1D adjusting the pH to ˜10-11 (CNC-AA not pH mediated); a sample where the dye methylene blue (MB) was coupled to CNC via the applicant's pH mediated protocol (CNC-MB pH mediated) as a negative control; a sample of the free dye azure A which has undergone pH meditated change without CNC (AA pH); a sample of the free dye methylene blue (MB), and an untreated sample. Different concentrations of these samples (6.25 ppm=unfilled; 12 ppm=checkered, 25 ppm=shaded; and 50 ppm=hatched) were irradiated for 10 minutes under ambient light using an LED set-up.

As shown in FIG. 10, light irradiation of the untreated sample presents no photobacteriocidal activity as demonstrated by the lack of killing (the log kill is almost zero). Importantly, at identical concentration tested, the pH mediated protocol for attachment of the dye azure A to CNC developed by the inventors (FIG. 1D) increases by approximatively 3 times the photobactericidal potency of CNC-AA compared to no pH mediated attachment or the free dye in solution. This result is new and unexpected.

CNC-AA Formulations

As indicated above, the inventors have shown that by supplementing CNC-AA concentrate to aqueous solutions, an effective photobiocidal disinfectant with broad-spectrum activity can be provided. The disinfectant solution can be sprayed directly on hard surfaces without staining them, providing thus a novel means for sanitization of common hard surfaces. In some embodiments the disinfectant can be prepared at a concentration within the range of approximately 1 mg to 200 mg of CNC-AA per liter of water.

Due to its aqueous compatibility, CNC-AA concentrate can easily be supplemented to other media, conferring to the latter the ability to produce singlet oxygen upon activation under ambient light. By way of examples, in some embodiments CNC-AA may be incorporated in film-forming polymers, such as paints, and hydrogels.

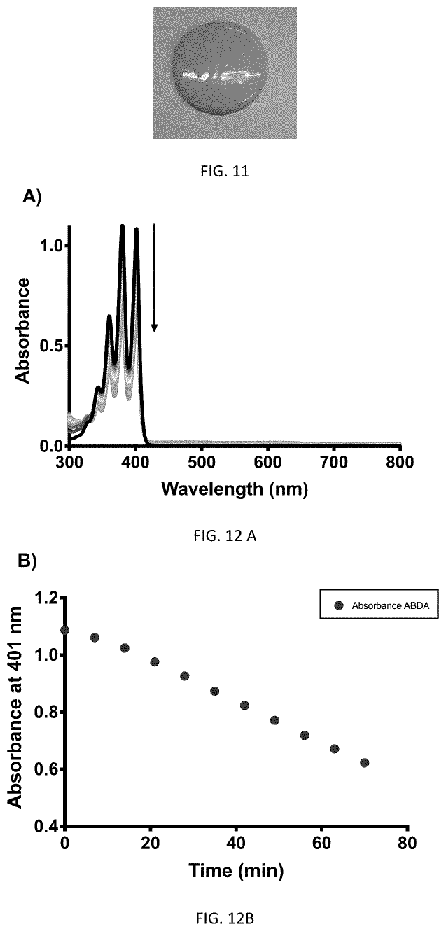

To illustrate the paint application, the inventors prepared a paint sample containing CNC-AA. Paint media was selected containing either acrylic or latex as the film-forming polymer, as these paints are known to adhere to a wide range of substrates, to be water resistant once dried, and to satisfy the regulation for low volatile organic compound (VOC) emission. A sample of CNC-AA was added to sample of paints, which were mixed with an overhead mechanical stirrer until homogeneous (FIG. 11). The paint prototype of FIG. 11 was very blue in color as a very high weight percent of CNC-AA (200 mg/L) was added to the base paint. The inventors' intent was to investigate the blending of CNC-AA to the paint and a higher stock concentration was necessary in order to visually assess homogeneous dispersion. However, such a high CNC-AA content is not necessary for an effective photobiocidal effect, at least in solution (FIGS. 7-9). Different weight percent loading will drastically change the hue of the paint.

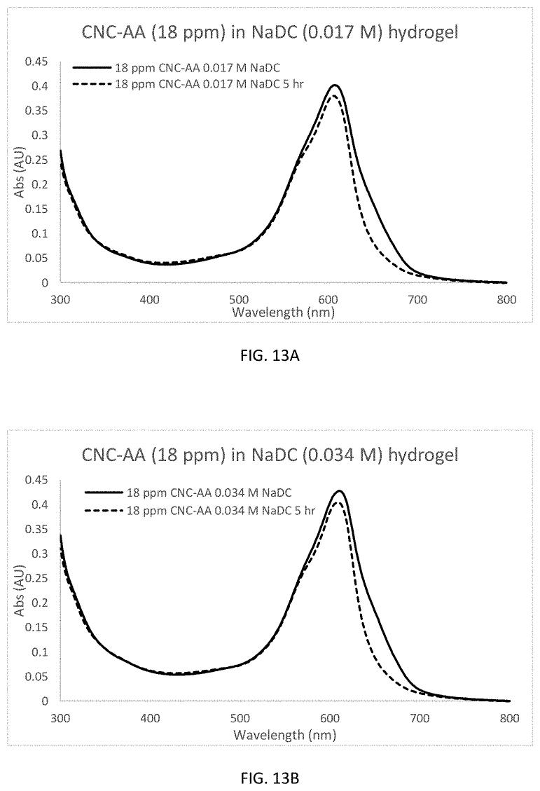

With a prototype in hand, the inventors investigated the ability of the paint to produce singlet oxygen. Herein, the inventors used an indirect methodology where a singlet oxygen sensor is used to detect the presence of singlet oxygen via spectroscopic techniques. The sensor presents a very specific absorption spectrum which is characterized by 5 sharp absorption bands in the UVA region between 300 nm and 400 nm (FIG. 12A). Upon chemical reaction with singlet oxygen, the sensor loses its ability to absorb in the 300 nm-400 nm region.^{15,39,40} Therefore, the production of singlet oxygen can be followed by monitoring the loss in the sensor absorbance (FIG. 12B). For this experiment, the inside of a 1.5 mL Eppendorf tube was initially coated with the CNC-AA paint (200 mg/L of paint) and left to dry overnight. The next day, the tube was filled with 1.5 mL of 1:1 RO:PBS (reverse osmosis: phosphate buffer saline 1×), which was left to equilibrate for 14 minutes. The solution was then removed and discarded. This step insured removal of any paint particles that wouldn't remain on the surface. The Eppendorf tube was subsequently rinsed out with reverse osmosis water (3×). Next, the Eppendorf tube was filled with 1 mL of 1:1 RO:PBS (1× buffer), containing the singlet oxygen sensor (FIG. 12). As can be seen in FIG. 12, CNC-AA in the paint form is still able to produce singlet oxygen upon exposure to ambient light with a cut-off filter for wavelengths below 405 nm. The filter was used in order to insure the sensor was not irradiated and its disappearance was actually due to the formation of singlet oxygen.

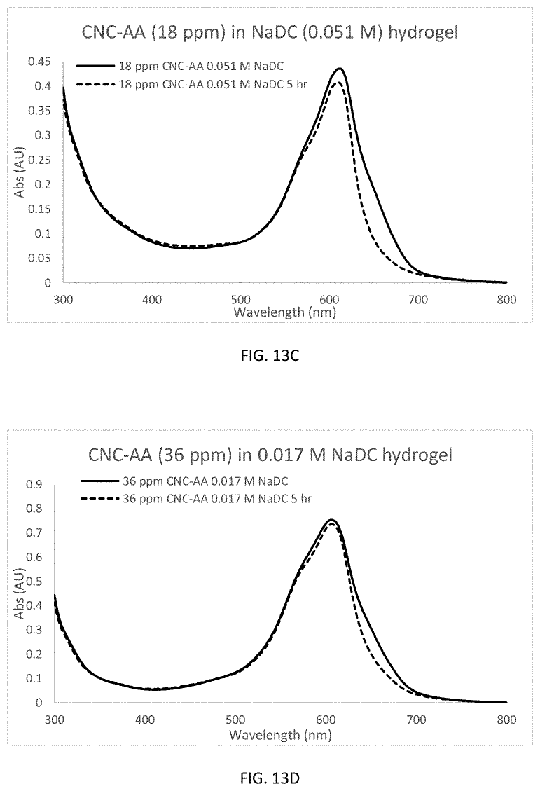

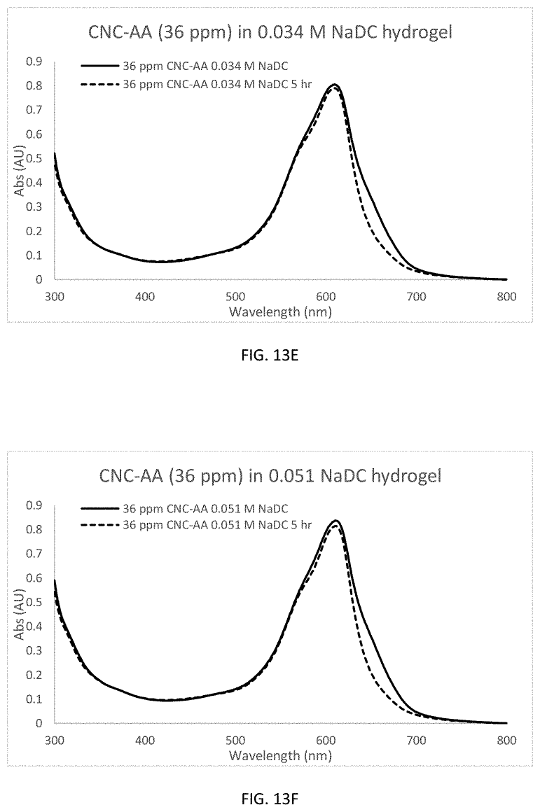

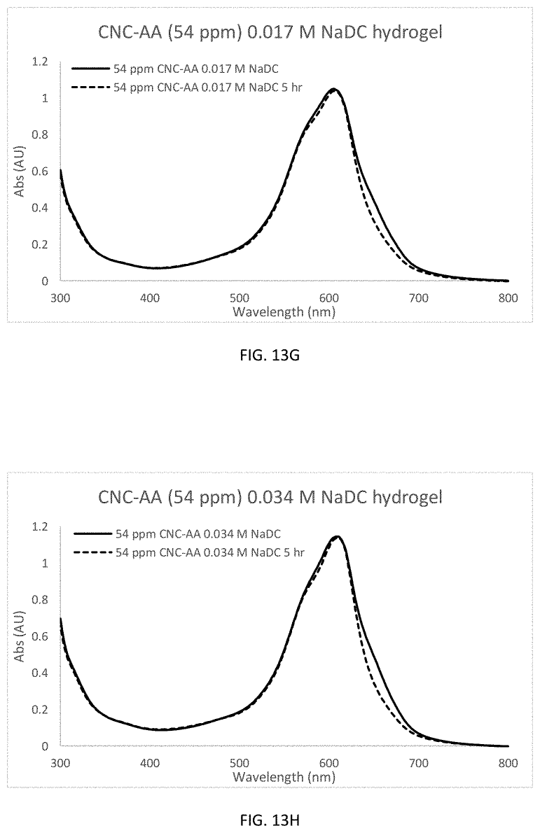

The inventors have also demonstrated that CNC-AA can be incorporated in hydrogels in some embodiments. A protocol for reproducibly forming sodium deoxycholate (NaDC) hydrogels with CNC-AA was as follows. An aliquot (270 μL, 540 μL or 810 μL) of a 0.02% (wt/V) solution of CNC-AA in PBS (1×) was added to a vial. Following this addition was 100 μL of saturated aqueous sodium chloride, an aliquot (200 μL, 400 μL or 600 μL) of 10% wt/v of NaDC in RO water. Finally, the mixture was diluted to 3 mL with addition of 0.1 M potassium phosphate buffer (pH 7). Prior to leaving the samples to gel, they were mixed to homogeneity. While the gels started to form within minutes, the samples were left overnight to ensure maximum gelation. In total, nine gels were prepared (FIGS. 13A-13I), all of which had indistinguishable strength by mechanical manipulation.

The inventors used the above protocol to prepare hydrogels within 3 mL plastic cuvettes. The photostability of each gel at each concentration of either sodium deoxycholate or CNC-AA were investigated. With reference to FIGS. 13A-13I, the inventors took an initial absorption spectrum, which shows the characteristic shift of the absorption to lower wavelengths compared to the free dye, and then exposed the gels to ambient light for 5 hours. The spectral sets for each sample (at t=0 hrs and t=5 hrs) are set forth in FIGS. 13A-13I. From this data, it looks like photobleaching or loss of absorbance is minimally occurring as there is almost no change in peak intensity, a small change in the maximum wavelength and only minor changes in the spectral features in the spectra (perhaps due to reorganization of the CNC-AA).

After establishing stable gels could be formed with the CNC-AA homogeneously dispersed therein, the inventors investigated if singlet oxygen production could be detected. In order to do this, the inventors carried out the indirect ABDA assay by incorporating ABDA into the gel. The protocol for inclusion of ABDA was as follows. Added to a plastic UV-vis cuvette was 540 μL of a 0.02% (wt/v) solution of CNC-AA in PBS (1×). This was followed by 100 μL of saturated aqueous sodium chloride, 200 μL of 10% (wt/v) of NaDC in RO water, 2160 μL of 0.1 M phosphate buffer (pH 7) and finally 30 μL of a 0.01 M ABDA solution in water. This was mixed and left to gel in the dark overnight.

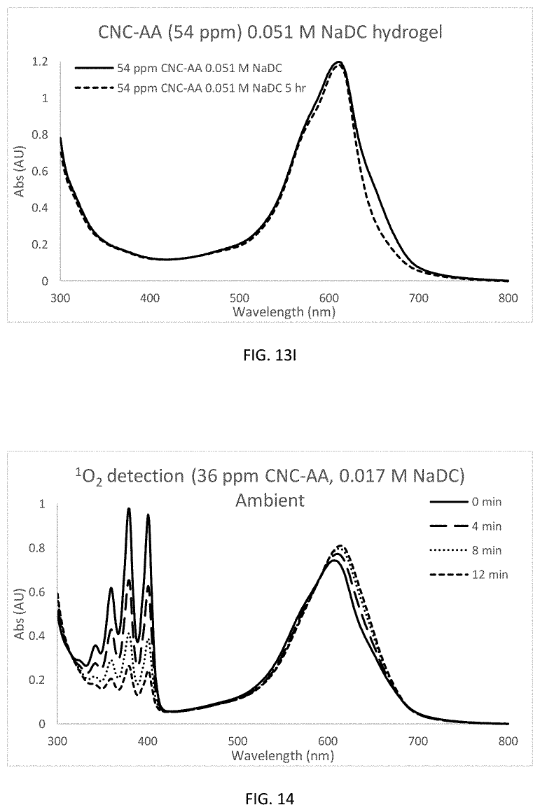

The inventors tried the assay under ambient light conditions. As can be shown in FIG. 14, the absorption of the probe ABDA between 300 and 400 nm decreased significantly with time (0 to 14 minutes) indicating the production of singlet oxygen in sodium deoxycholate hydrogels comprising CNC-AA.

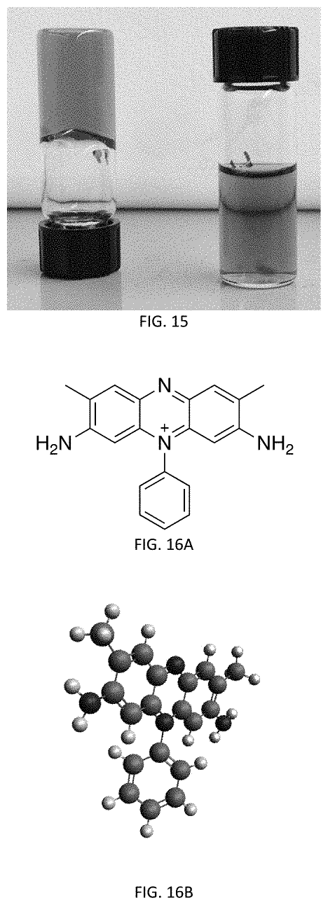

Further, the inventors have determined that CNC-AA can also be homogenously dispersed in gelatin, forming a strong gel (FIG. 15). Gelatine is particularly interesting for potential wound healing applications. As indicated below, CNC-AA has been shown to be non-irritating to human tissue at biologically effective concentrations.

Additionally, the inventors demonstrated that CNC-photosensitizer conjugates can be homogenously distributed within other biocompatible or biomedical hydrogels that are commonly found in medical application for wound dressings.^{66,67} In some embodiments, the biocompatible or biomedical hydrogel is hydroxyethyl cellulose, sodium carboxymethyl cellulose, sodium polyacrylate or combinations thereof. Other examples of biocompatible or biomedical hydrogels can be found in Sannino et al. (2009) or Calo et al. (2015), the entire contents of which are incorporated herein by reference.

In some embodiments, formulations comprising the CNC-photosensitizer conjugates distributed in biocompatible or biomedical hydrogels are used as disinfectants and/or antiseptics for wounds, such as skin wound, surface wounds or open wounds. As used herein, “skin wound” or “surface wound” refers to a superficial wound on the surface of the skin and “open wound” refers to an exposed wound or a wound on an exterior portion of an organ to which medical formulations can be applied onto. Example surface wounds include, but are not limited to: skin wounds (i.e. cuts or incisions), ulcers (such as diabetic or venous leg ulcers), or burns.

The inventors were able to incorporate both CNC-AA and CNC-TB in concentrations ranging from 0 to 0.1% (wt/v) in hydrogel formulation based upon the gelators hydroxyethyl cellulose, sodium carboxymethyl cellulose, sodium polyacrylate or combinations thereof with and without addition of propylene glycol in RO water or PBS. In some embodiments, the CNC-photosensitizer conjugates are distributed in biocompatible hydrogels at a concentration of 0.01-10% wt/v, 0.01-5% wt/v, 0.01-1% wt/v, 0.01-0.5% wt/v, or 0.01-0.1% wt/v.

The gels were created by adding the correct amount of gelator to prepare the desired % wt/v, following by addition of propylene glycol (if needed) along with the appropriate amount of PBS (1×) solution and RO water to make up the final volume. The mixtures were stirred for 2 hours while the gelator hydrate and a thick hydrogel formed. In the case of sodium polyacrylate, the gelator was vigorously stirred in RO water until the powder had dispersed and the mixture was neutralized with 0.1 M NaOH, at which point gelation occurred.

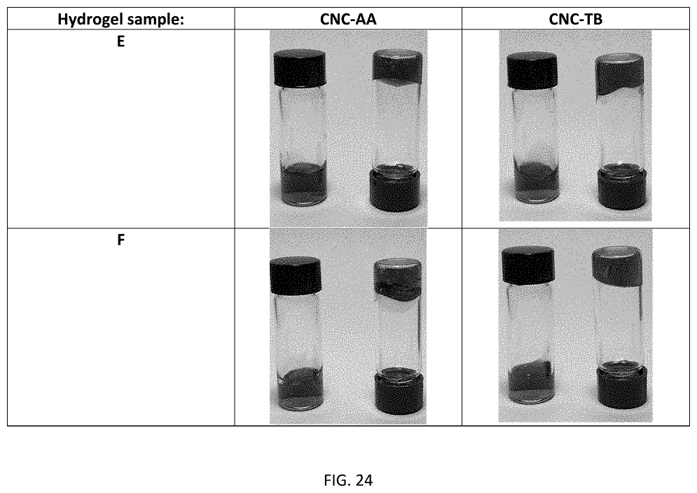

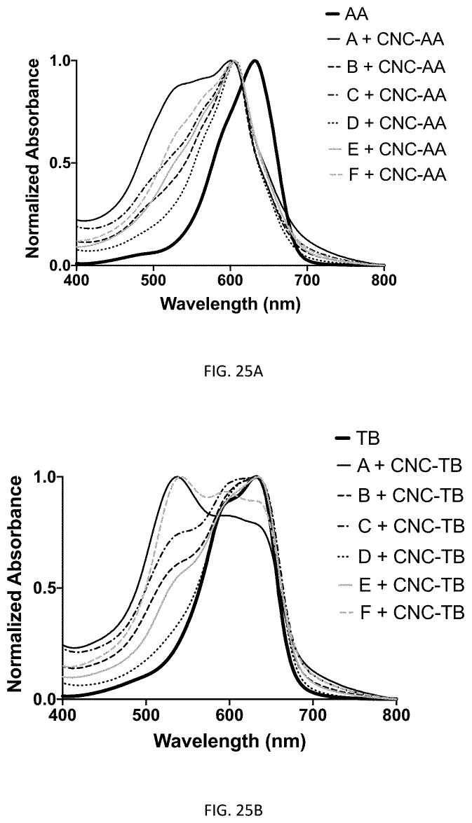

The inventors added CNC-photosensitizer conjugates, such as CNC-AA or CNC-TB to the formulation either prior to hydrogel formation or after the hydrogels had formed. If added after the hydrogels had formed, the desired volume of CNC-AA or CNC-TB was deposited on the hydrogel, which was then mechanically mixed to obtain the desired formulation. Both method of incorporation resulted in hydrogels where the CNC-photosensitizer conjugates, such as CNC-AA or CNC-TB, was homogenously dispersed with mechanical property typical for an hydrogel upon inversion as seen in FIG. 24.

The inventors showed that CNC-AA and CNC-TB present the characteristic shift of the absorption to lower wavelengths compared to the free dye once homogenously dispersed in these new hydrogels formulations with a potential to be used as wound dressing (FIGS. 25A-25B).

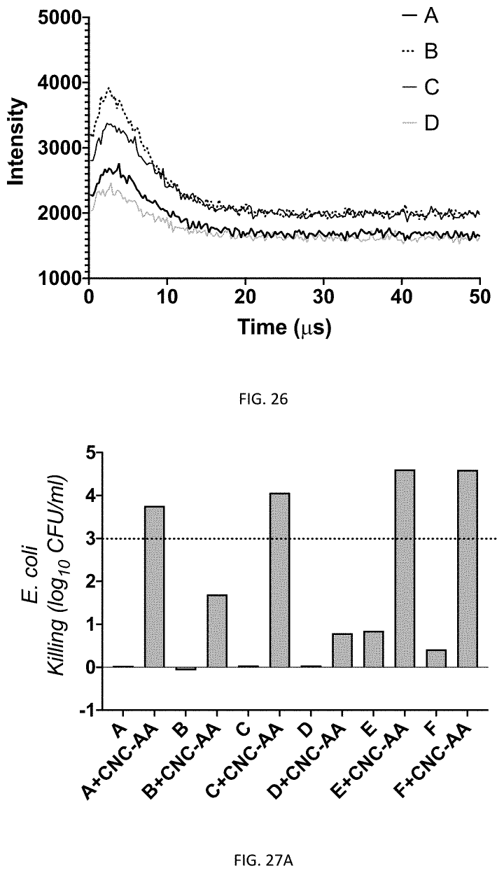

The inventors tested the ability of these new hydrogels containing the CNC-photosensitizer conjugates to produce singlet oxygen. The inventors used the direct detection of singlet oxygen luminescence at 1270 nm. The latter was detected via the customized instrument described above. As shown in FIG. 26, the characteristic singlet oxygen emission signal was obtained for all hydrogel formulations containing CNC-AA. These results demonstrate the ability to the new hydrogels containing the CNC-photosensitizer conjugates to produce singlet oxygen upon light irradiation.

Further the inventors tested the photobiocidal efficacy of the new hydrogel formulations containing CNC-photosensitizer conjugates, either CNC-AA or CNC-TB, against both Gram-positive and Gram-negative bacteria via two different methodologies. The inventors use both an inhibition zone method as well as a direct testing of the hydrogels to assess the antimicrobial ability of the hydrogels upon ambient light exposure.

The inhibition zone method required to spread evenly onto nutrient agar plates 80 μL of 10⁷-10⁸ CFU/mL suspensions of either E. coli or S. aureus grown in LB media and subsequently suspended in PBS (1×). Onto the inoculated plates was spread 100 μL of each candidate gel and respective control gels. Spreading was carried out with sterile applicators and carefully controlled to cover the surface area of a templated circle of known diameter equal to 17 mm. The prepared agar plates were subjected to irradiation with white ambient light. The plates were incubated for 24 hours at 37° C. prior to measuring relative growth and size of inhibition zones.

The direct method required a 2:15 (v:v) ratio of bacterial suspension (10⁷-10⁸ CFU/mL, 1×PBS) to be mixed with the candidate hydrogel were mixed in 24-well culture plates using flame-sterilized nichrome wire. Then, the 24-well plate was irradiated with white ambient light for 20 minutes. Hydrogels containing CNC-photosensitizer conjugates were directly compared to respective control formulations tested under the same conditions. Following irradiation, the entire assay volume was quantitatively diluted with the appropriate volume of PBS (1×) for a tenfold dilution. Tenfold serial dilutions of the recovered inoculated hydrogel were prepared in PBS (1×). To determine the survival fraction following irradiation, 10 μL aliquots of each serial dilution were plated on nutrient agar by spreading this volume (gravity) over a lane measuring 9 cm. The plates were incubated for 24 hours at 37° C. and colony counts were performed.

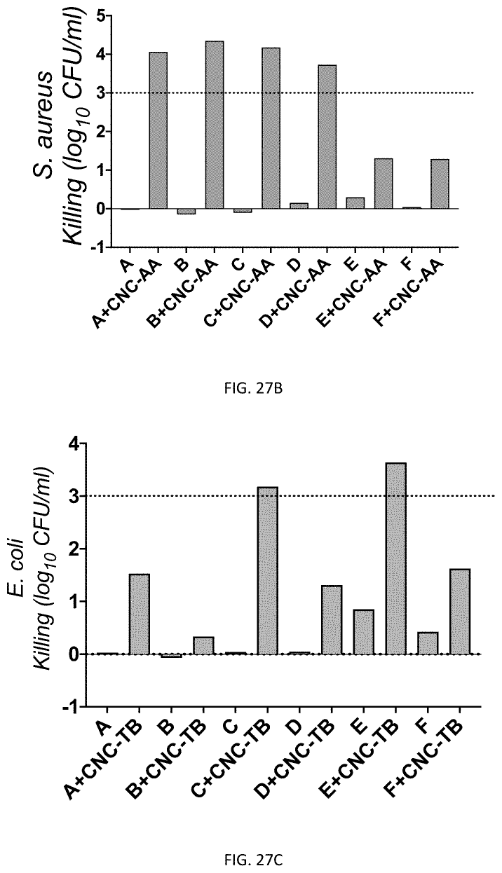

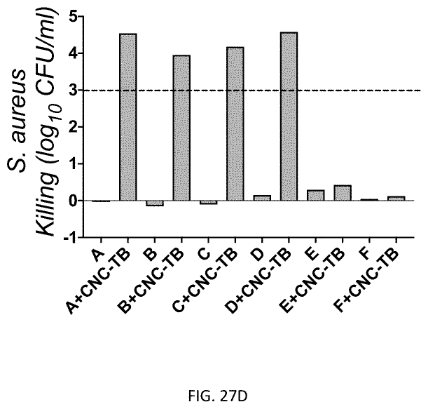

The results are new and exciting as all the hydrogels used in wound dressing applications and tested herein show strong antimicrobial activity upon light exposure against both Gram-positive and Gram-negative bacterial strains only when they contain the CNC-photosensitizer conjugates, CNC-AA or CNC-TB ranging in concentration from 0.01% to 0.1% wt/v (FIGS. 27A-27B-27C-27D).

Table 1 below summarizes the antimicrobial activity upon against both E. coli and S. aureus light activation with fluorescent light of different concentration of CNC-AA or CNC-TB in hydrogels sample containing: (A) hydroxyethyl cellulose and propylene glycol in reverse osmosis water; (B) hydroxyethyl cellulose and propylene glycol in reverse osmosis water; (C) hydroxyethyl cellulose, carboxymethyl cellulose and propylene glycol in reverse osmosis water; (D) carboxymethyl cellulose and propylene glycol in reverse osmosis water; (E) sodium polyacrylate type I in reverse osmosis water; (F) sodium polyacrylate type II in reverse osmosis water. The data were obtained via the inhibition zone method. Relative growth was assessed as G (microbial growth nearly indistinguishable from the rest of plate), PG (partial growth) and NG (no growth). Inhibition zone was calculated from an average of the vertical and horizontal cross-sectional diameters of affected growth zones as measured with a ruler.

TABLE 1

| | | Growth | Inhibition diameter |
|---|---|---|---|
| Gel Sample | Microorganism | Level | (mm) |
| A | E. coli | G | — |
| A + 0.01% CNC-AA | E. coli | NG | 15.8 |
| A + 0.1% CNC-AA | E. coli | NG | 18.3 |
| A + 0.01% CNC-TB | E. coli | NG | 16.8 |
| A + 0.1% CNC-TB | E. coli | NG | 19 |
| A | S. aureus | G | — |
| A + 0.01% CNC-AA | S. aureus | G | — |
| A + 0.1% CNC-AA | S. aureus | NG | 18 |
| A + 0.01% CNC-TB | S. aureus | PG | 13.5 |
| A + 0.1% CNC-TB | S. aureus | NG | 19.5 |
| B | E. coli | G | — |
| B + 0.01% CNC-AA | E. coli | NG | 15.8 |
| B + 0.1% CNC-AA | E. coli | NG | 18.3 |
| B + 0.01% CNC-TB | E. coli | NG | 16.8 |
| B + 0.1% CNC-TB | E. coli | NG | 19 |
| B | S. aureus | G | — |
| B + 0.01% CNC-AA | S. aureus | G | — |
| B + 0.1% CNC-AA | S. aureus | NG | 18 |
| B + 0.01% CNC-TB | S. aureus | PG | 13.5 |
| B + 0.1% CNC-TB | S. aureus | NG | 19.5 |
| C | E. coli | G | — |
| C + 0.01% CNC-AA | E. coli | NG | 13.8 |
| C + 0.1% CNC-AA | E. coli | NG | 17.8 |
| C + 0.01% CNC-TB | E. coli | NG | 14 |
| C + 0.1% CNC-TB | E. coli | NG | 18.3 |
| C | S. aureus | G | — |
| C + 0.01% CNC-AA | S. aureus | G | — |
| C + 0.1% CNC-AA | S. aureus | NG | 17 |
| C + 0.01% CNC-TB | S. aureus | NG | 12.3 |
| C + 0.1% CNC-TB | S. aureus | NG | 17 |
| D | E. coli | PG | 14 |
| D + 0.01% CNC-AA | E. coli | NG | 14 |
| D + 0.1% CNC-AA | E. coli | NG | 18.8 |
| D + 0.01% CNC-TB | E. coli | NG | 14 |
| D + 0.1% CNC-TB | E. coli | NG | 18.8 |
| D | S. aureus | G | — |
| D + 0.01% CNC-AA | S. aureus | PG | 11.5 |
| D + 0.1% CNC-AA | S. aureus | NG | 18.8 |
| D + 0.01% CNC-TB | S. aureus | PG | 12.8 |
| D + 0.1% CNC-TB | S. aureus | NG | 19 |
| E | E. coli | G | — |
| E + 0.01% CNC-AA | E. coli | PG | 16 |
| E + 0.1% CNC-AA | E. coli | NG | 17.8 |
| E + 0.01% CNC-TB | E. coli | NG | 15.8 |
| E + 0.1% CNC-TB | E. coli | NG | 18 |
| E | S. aureus | G | — |
| E + 0.01% CNC-AA | S. aureus | G | — |
| E + 0.1% CNC-AA | S. aureus | NG | 18 |
| E + 0.01% CNC-TB | S. aureus | PG | 14.3 |
| E + 0.1% CNC-TB | S. aureus | NG | 17.8 |
| F | E. coli | G | — |
| F + 0.01% CNC-AA | E. coli | PG | 19 |
| F + 0.1% CNC-AA | E. coli | NG | 20 |
| F + 0.01% CNC-TB | E. coli | NG | 15.5 |
| F + 0.1% CNC-TB | E. coli | NG | 18 |
| F | S. aureus | G | — |
| F + 0.01% CNC-AA | S. aureus | G | — |
| F + 0.1% CNC-AA | S. aureus | NG | 18.8 |
| F + 0.01% CNC-TB | S. aureus | PG | 14.5 |
| F + 0.1% CNC-TB | S. aureus | NG | 16 |

Compatibility of CNC-AA with Test Materials

Formulations comprising CNC-AA are compatible with a wide variety of materials and substrates, including metals and glass. According to one testing protocol, a sample of the CNC-AA composition was added to a beaker of PBS (100 ppm in 1×PBS). The test material was placed in the same beaker. The beaker was left in ambient light and the sample was left to stand in solution for 1 hour. As a comparison, a different sample of the same material was placed in a beaker of commercial bleach (titrated and found to be 6.25%). Each comparison test was also carried out for 1 hour. Photographs were acquired prior to exposure, at initial exposure, after an hour in solution and finally after removal from solution. Samples were inspected visually for changes in color or corrosion.

The results, in brief, demonstrate that CNC-AA is compatible with many materials and substrates including metals and glass. In particular, CNC-AA was compatible with aluminum, zinc, vinyl rubber, brass, copper, blue PVC, grey polyethylene, satin nickel finish, stainless steel, black ABS, orange polyethylene, black rubber and black vinyl upholstery.

### Skin Compatibility of CNC-AA

The test material CNC-AA was also investigated in skin compatibility tests. EpiDerm human tissue model (EPI-200), produced by MatTek Corporation, was used to evaluate the skin irritation potential of CNC-AA. The test material was topically exposed to EpiDerm tissues as follows.

Briefly, EpiDerm tissues were removed from packaging. Each insert was placed in one well of a 6-well plate containing 0.9 mL EPI-100-NMM. The tissues were equilibrated at 37±1° C./5±1% CO2/90%±10% RH for 1 hour ±5 minutes. Following 1 hour equilibration, the EPI-200 tissues were transferred from upper wells into the lower wells of the 6-well plate containing 0.9 mL EPI-100 NMM media. The tissues were equilibrated at 37±1° C./5±1% CO2/90%±10% RH overnight (18±3 hours).

Following overnight equilibration, 30 μl of negative control (NC), positive control (PC) and test article (TA) (mesh was used for NC, PC and τΔ) were applied topically to n=3 tissues per treatment group. The treatments were performed at an interval of 1 minute between tissues and the tissues were incubated at 37±1° C./5±1% CO2/90%±10% RH for 35±1 minutes.

After 35 minutes, all plates were removed from the incubator and place into a biological safety cabinet until the 60-min exposure period was completed for the first dosed tissue. The tissues were rinsed with sterile DPBS (pH 7.0) by filling and emptying the tissue inserts 15 times. After the 15th rinse from washing bottle, the inserts were completely submerged 3 times in 150 ml DPBS (a new 150 mL DPBS was used for each dose group). Finally, the tissues were rinsed once from inside and once from outside with sterile DPBS. Excess DPBS was removed by gentle shaking of the insert, inserts were blotted and the tissues were transferred to 6-well plates pre-filled with 0.9 mL EPI-100-NMM media. The surface was carefully dried with a sterile swab. The tissues were incubated in an incubator for next 24±2 hours.

24 hours post-treatment the tissues were re-fed with 0.9 mL EPI-100-NMM medium and incubated at 37±1° C./5±1% CO2/90%±10% RH for an additional 18±2 hours. At the end of 18±2 hours post-incubation period the tissues were removed from culture and MTT analysis was performed.

MTT analysis to assess tissue viability was performed following the procedure developed by MatTek. Briefly, just prior to the end of 18 hours post-incubation period, 2 mL MTT concentrate was thawed (supplied by MatTek, part number MTT-100-CON) and added to 8 mL MTT diluent (supplied by MatTek, part number MTT-100-DIL) to prepare the MTT reagent. The reconstituted MTT reagent was protected from light by covering the tube with aluminum foil. 300 μl of the MTT reagent was dispensed into the appropriate number of wells of a 24-well plate and was equilibrated to 37° C. by placing the plate in a 37±1° C./5±1% CO2/90%±10% RH incubator. The inserts were placed into the wells containing the pre-warmed MTT reagent and incubated at 37±1° C./5±1% CO2/90%±10% RH for 3 hours ±5 min. Viable tissues converted the MTT to a purple dye. The amount of conversion is proportional to the viability of the tissue. 2 ml of extractant solution (supplied by MatTek, part number MTT-100-EXT) was pipetted into each well of a 24-well plate. At the end of the incubation, the tissues were removed from the MTT, blotted dry on a paper towel and moved to the plate containing 2 ml extractant solution. Extraction was performed for two hours at room temperature on a shaker. The plate was protected from light exposure and sealed to prevent extractant evaporation. At the end of the extraction period, the extractant solution was combined from the apical compartment with that in the well below, the tissue inserts were removed and discarded. The extractant solution was mixed well and 200 μl of each sample was added to a 96-well plate. Added 200 μl of sample to a second well in the 96-well plate and all samples were prepared in duplicate. The optical density (OD) of the extracted samples were determined at 570 nm using 200 μl of extractant as a blank using a spectrophotometer.

According to the above-described MTT assay, the test CNC-AA sample had tissue viability of 97.34% and was therefore classified as non-irritant.

### Additional CNC-Dye Conjugates

In addition to the CNC-AA embodiment, other CNC-dye conjugates have been prepared according to the pH-mediated protocol described above (FIG. 1D). In some embodiments, the photosensitizer molecules are Azure A, Azure B, Safranin O, Methylene Violet, 3RAX, Toluidine Blue 0 (referred to as Toluidine Blue), Thionine Acetate, Rhodamine 6G, Cresyl Violet, or Acriflavine. In some embodiments, the photosensitizer molecules are selected from the group consisting of azure A, azure B, toluidine blue, thionine acetate and cresyl violet. In one embodiment, the photosensitizer molecules are azure A.

In some embodiments the concentration of photosensitizer molecules in the composition is within the range of 0.01 to 0.1 mmol/100 mg. In some embodiments the concentration of photosensitizer molecules in the composition is within the range of 0.03 to 0.075 mmol/100 mg. In some embodiments, the concentration of photosensitizer molecules in the composition is about 0.05 mmol/100 mg.

All of the alternative dyes are photosensitizer molecules that can be coupled to the surface of CNC, as demonstrated by elemental analysis indicating the concentration of dye attached per 100 mg of CNC. All of the tested dyes comprise acidic protons. Coupling of the dyes on the surface of CNC can be observed spectroscopically by a shift of the absorption of the dye to lower wavelengths in reverse osmosis (RO) water, with the exception of two dyes discussed below (rhodamine 6G and acriflavine). Furthermore, experimental data with multiple dyes suggests that the planarity of the dye is important to molecular organization (or packing). Improved packing during the adsorption process likely leads to better retention of the dye on the surface of CNC when the CNC-dyes are suspended in phosphate buffer (PBS). Finally, the greater retention of the dye on the surface of CNC in phosphate buffer can be correlated to an increased antimicrobial efficacy of the CNC-dye conjugate compared to the free dye in phosphate buffer.

The pH mediated protocol to attach the alternative photosensitizer dyes on the surface of CNC is identical to the one discussed above for CNC-AA (FIG. 1D). Briefly, the protocol involves: suspension of [O]CNC at a 1% wt/v concentration; addition of a dye photosensitizer; modifying the pH to ˜10-11 with the addition of 1 M NaOH, stirring at room temperature overnight; carefully adjusting the pH to ˜1 to acidify the mixture; collection and crude purification of samples via centrifugation techniques; final purification via overnight Soxhlet distillation in ethanol; and finally, evaporation of ethanol to leave the dry CNC-dye for collection.

Table 2 below lists the dyes used and the short form name given to each CNC-dye conjugate. For comparison, a data set for one of the batches of CNC-AA is also included.

TABLE 2

| Dye | Abbreviatio |
|---|---|
| Azure A | CNC-AA |
| Safranin O | CNC-SO |
| Methylene Violet | CNC-MLV |
| Azure B | CNC-AB |
| Toluidine Blue O | CNC-TB |
| Thionine Acetate | CNC-TA |
| Rhodamine 6G | CNC-R6G |
| Cresyl Violet | CNC-CV |
| Acriflavine | CNC-AF |

These samples were all submitted for elemental analysis (EA) to determine the percent nitrogen present in each material. As indicated above, the photosensitizer dyes are the only species with nitrogen so the amount of dye per gram of CNC material can thus be back calculated. Each sample was subjected to two EA runs and the average mmol of dye per 100 mg of sample is presented in the Table 3 below.

TABLE 3

| | | mmol dye in |
|---|---|---|
| Sample | % N | 100 mg of CNC |
| CNC-AA | 1.89 | 0.0451 |
| CNC-SO | 2.53 | 0.0451 |
| CNC-MLV | 2.25 | 0.0401 |
| CNC-AG | 2.30 | 0.0546 |
| CNC-TB | 2.43 | 0.0577 |
| CNC-TA | 3.08 | 0.0732 |
| CNC-R6G | 0.325 | 0.0116 |
| CNC-CV | 2.53 | 0.0680 |
| CNC-AF | 1.34 | 0.0318 |

For each CNC-dye conjugate prepared, initial spectroscopic characterizations of the conjugate and individual dyes were carried out. These are detailed in the graphs presented in FIGS. 4B, 16C, 17C, 18C, 19C, 20C, 21C, 22C and 23C. Each graph summarizes experimental results for the CNC-dye conjugate in phosphate buffer saline solution at pH 7.4 (used for the biological assays); the CNC-dye conjugate in reverse osmosis (RO) water; and the respective free dye free in PBS and in RO water. The chemical structure and shape of each dye is also discussed below for each example.

With reference to the chemical structure shown in FIGS. 1B and 1C, the azure A molecule contains a primary amine (NH2), which is important for the pH mediated protocol. The azure A molecule is known to have a planar conformation. As discussed above, the planarity of the molecule appears to be important in respect of the packing of the photosensitizer adsorbed on to the surface of the CNC. As shown in FIG. 4B, coupling of the dye can be observed spectroscopically by a shift of the absorption of the dye to a lower wavelength. When CNC-AA conjugate is suspended in PBS buffer, a change is observed, but the dye retains a maximum absorption which is significantly different than the free dye in solution. As described above, CNC-AA is significantly more effective at killing bacteria than the free dye, AA.

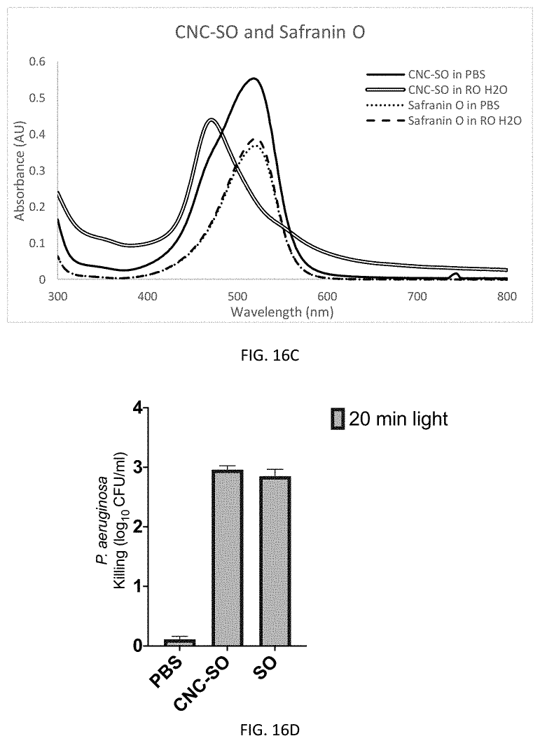

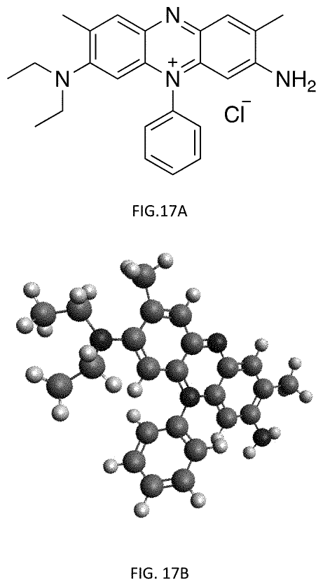

With reference to FIGS. 16A-16D, a CNC conjugate comprising the dye safranin O (SO) was synthesized and characterized. As shown in FIGS. 16A and 16B, SO has a non-planar conformation; the phenyl ring substituent is twisted compared to the primary plane of the molecule. A shift towards shorter wavelengths is observed when CNC-SO powder is dispersed in RO water (FIG. 16C). However, the absorption of the CNC-dye shifts back to the absorption of the free dye in solution when dispersed into PBS. This result suggests that the shape (planarity) of the photosensitizer plays a role in the packing and thus stability of the photosensitizer adsorbed onto the surface of the CNC. When the antimicrobial activity of the CNC-SO was tested after 20 minutes of light exposure and compared to the activity of the free SO dye in PBS solution, the two samples did not show a difference in antimicrobial efficacy (against P. aeruginosa) within experimental errors (FIG. 16D). The CNC-SO concentration used in the antimicrobial test was 100 ppm. The concentration of SO was adjusted to match the absorption value of the CNC-SO sample.

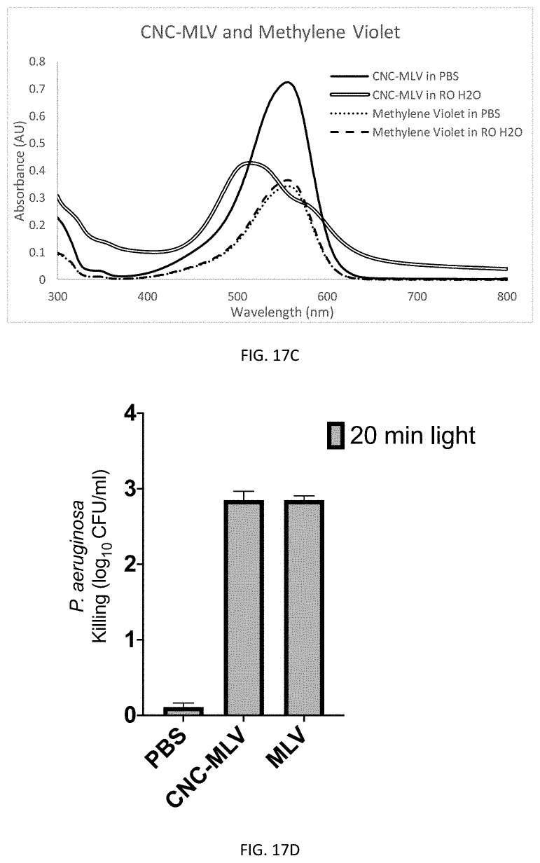

With reference to FIGS. 17A-17D, a CNC conjugate comprising the dye methylene violet 3RAX (MLV) was synthesized and characterized. The chemical structure of MLV is shown in FIGS. 17A and 17B. Similar to the dye SO described above, MLV has a non-planar conformation. Coupling of MLV was confirmed by the shift toward lower wavelengths (FIG. 17C). However, the adsorption of the MLV onto the surface of the CNC was not stable as shown in PBS where the absorption of the CNC-MLV was similar to the absorption profile of the free MLV. Similar to the case with SO, no difference in antimicrobial efficacy (against P. aeruginosa) of CNC-MLV compared to free MLV was seen within experimental errors (FIG. 17D) after 20 minutes of light exposure. The CNC-MLV concentration used in the antimicrobial test was 100 ppm. The concentration of free MLV was adjusted to match the absorption value of the CNC-MLV sample.

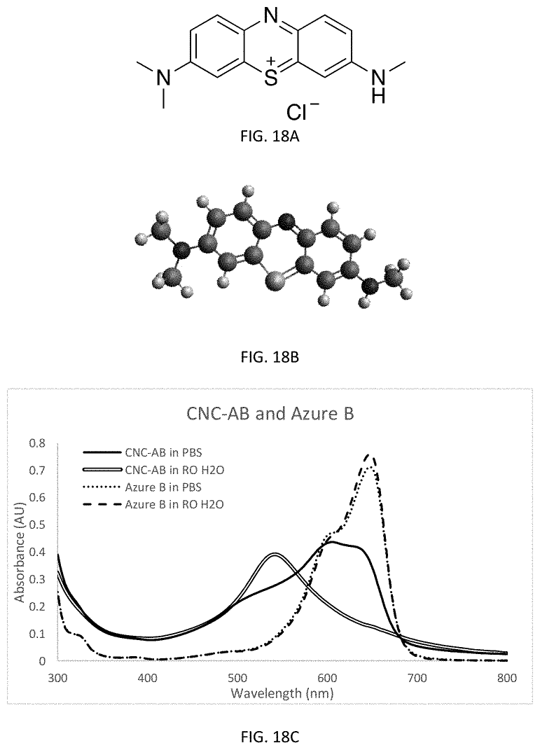

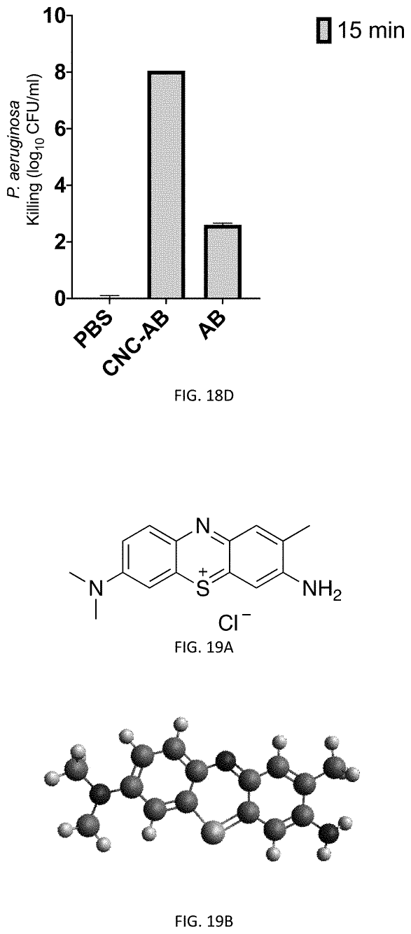

With reference to FIGS. 18A-18D, a CNC conjugate comprising the dye azure B (AB) was synthesized and characterized. The chemical structure of azure B is shown in FIGS. 18A and 18B. Azure B is very similar in structure to azure A except for the presence of a secondary amine instead of a primary amine. As shown in FIG. 18C, the attachment of the AB dye was successful via the pH mediated protocol described above, as a shift to a lower wavelength was observed in RO water. However, once the CNC-AB composition was dispersed in PBS a partial loss of the photosensitizer in solution was observed. Some degree of retention of the dye on the surface of the CNC was observed by the shoulder between 500 nm and 600 nm (FIG. 18C). As shown in FIG. 18D, CNC-AB was more effective at killing bacteria (P. aeruginosa) than the free AB. The concentration of the CNC-AB used in the antimicrobial test was 100 ppm and the concentration of free AB was selected to have an equivalent optical density.

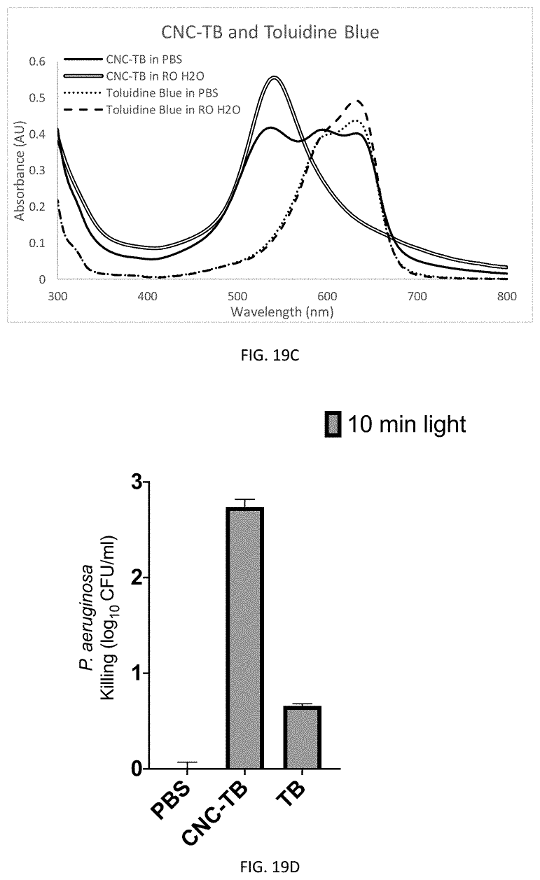

With reference to FIGS. 19A-19D, a CNC conjugate comprising the dye toluidine blue (TB) was synthesized and characterized. The chemical structure of toluidine blue is shown in FIGS. 19A and 19B. Toluidine blue is a planar molecule with a primary amine similar to azure A. Unlike azure A, toluidine blue comprises a bulky methyl functional group in proximity of the primary amine. The spectroscopic data (FIG. 19C) indicated successful attachment of TB to CNC via the pH mediated protocol described above as a shift to a lower wavelength was observed in RO water. However, the methyl functional group in proximity of the primary amine appears to impact the stability of adsorption of the dye on the surface of CNC, as a partial loss of the TB dye to the solution occurred upon suspension in PBS buffer. The CNC-TB efficacy was tested against bacteria. As shown in FIG. 19D, CNC-TB was more effective at killing bacteria (P. aeruginosa) than the free TB after 10 minutes of light exposure. The concentration of the CNC-TB used in the antimicrobial test was 6.25 ppm and the concentration of free TB was selected to match the absorption of CNC-TB.

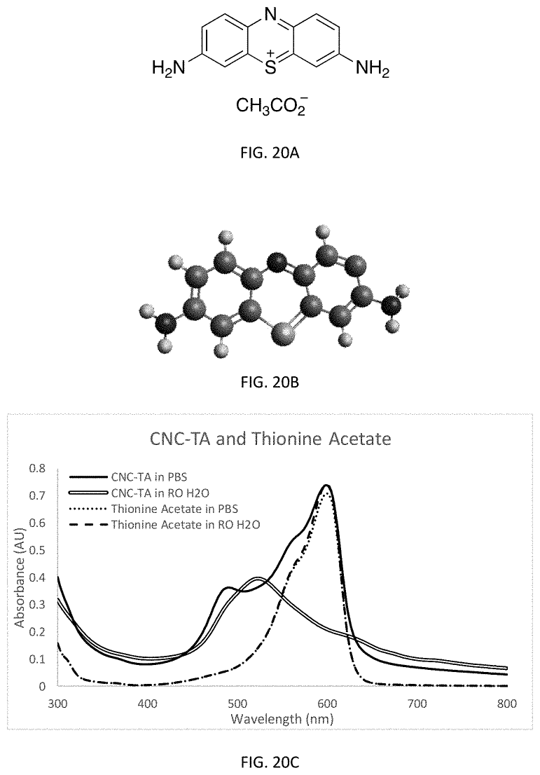

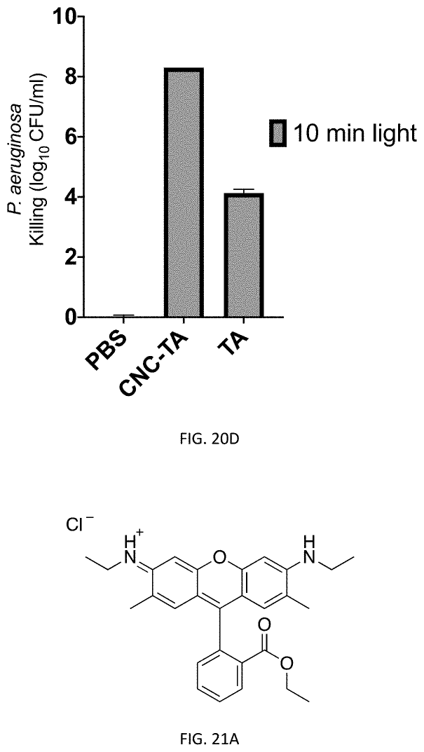

With reference to FIGS. 20A-20D, a CNC conjugate comprising the dye thionine acetate (TA) was synthesized and characterized. The chemical structure of thionine acetate is shown in FIGS. 20A and 20B. Thionine acetate has two primary amines in contrast to the one amine present in azure A. Although the molecule is planar, the adsorption of TA on the CNC surface was not as stable as for azure A as a partial loss of the TA dye from the CNC surface to the free dye was observed (FIG. 20C). This could possibly be attributed to the greater hydrophilicity of the TA dye. As shown in FIG. 20D, CNC-TA was more effective at killing bacteria (P. aeruginosa) than the free TA dye after 10 minutes of light exposure. The concentration of the CNC-TA used in the antimicrobial test was 6.25 ppm and the concentration of free TA was selected to match the maximum absorbance of CNC-τΔ.

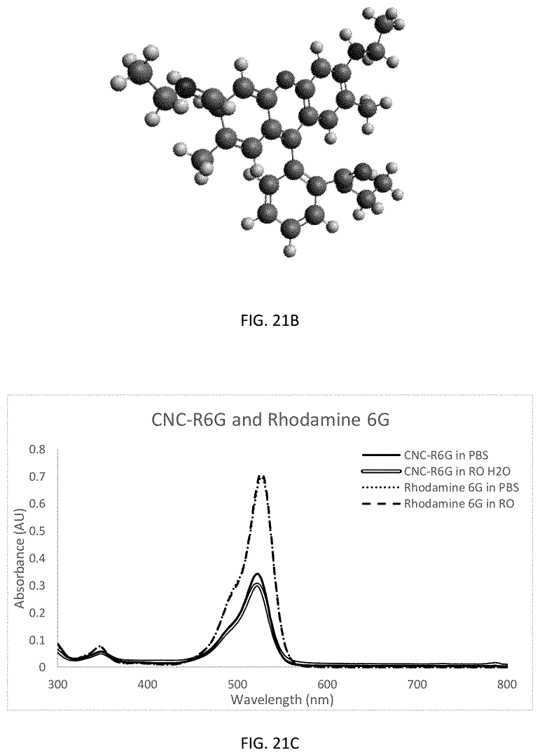

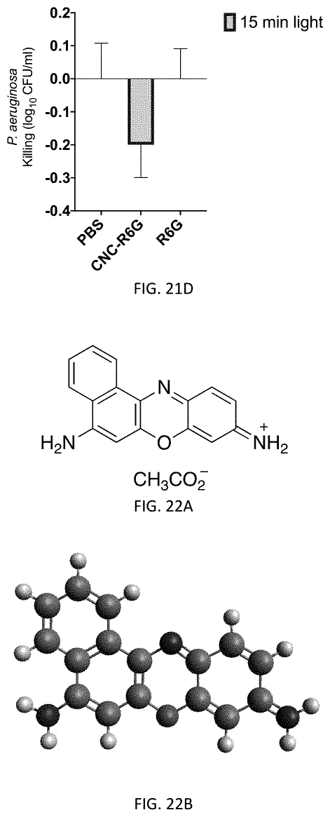

With reference to FIGS. 21A-21D, a CNC conjugate comprising the dye rhodamine 6G (R6G) was synthesized and characterized. The structure of rhodamine 6G is shown in FIGS. 21A and 21B. The dye is non-planar, it is very bulky and doesn't present any primary amine. While dye adsorption on the surface of CNC can be observed via elemental analysis, it is very minimal with only 0.01 mmol of dye/100 mg of CNC (see Table 3 above). No shift in the absorption of the dye was observed upon coupling to CNC (FIG. 21C). Therefore, it may be postulated that the R6G dye was not effectively coupled to the CNC in this example. Further, no antimicrobial effect (against P. aeruginosa) was observed after 15 minutes of light exposure (FIG. 21D). The concentration of the CNC-R6G and free dye used in the antimicrobial tests were 100 ppm.

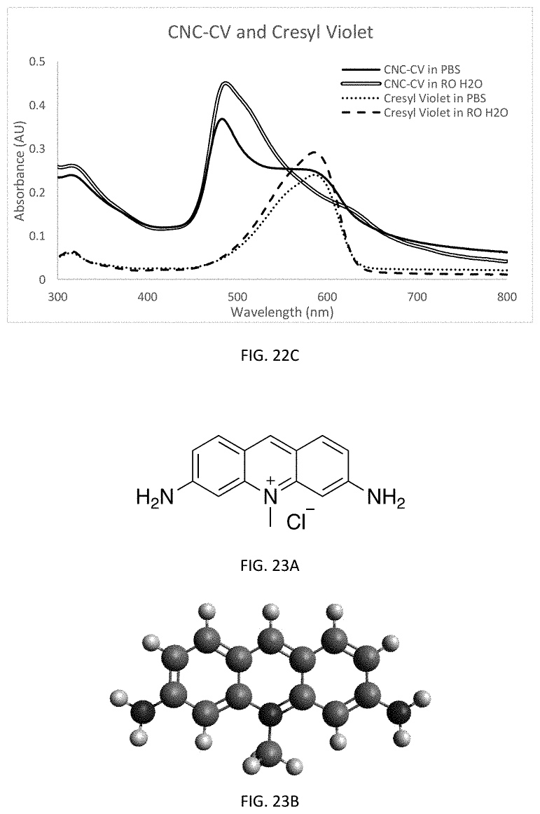

With reference to FIGS. 22A-22C, a CNC conjugate comprising the dye cresyl violet (CV) was synthesized and characterized spectroscopically. The structure of cresyl violet is shown in FIGS. 22A and 22B. Cresyl violet is a very planar dye, with two secondary amines. Spectroscopically, a shift to lower wavelength was observed, indicating that the CNC and CV were effectively coupled (FIG. 22C). Retention of the dye on the surface of the CNC appears to be partially lost when the conjugate is suspended in RO water (FIG. 22C), perhaps due to the hydrophilicity of the CV dye.

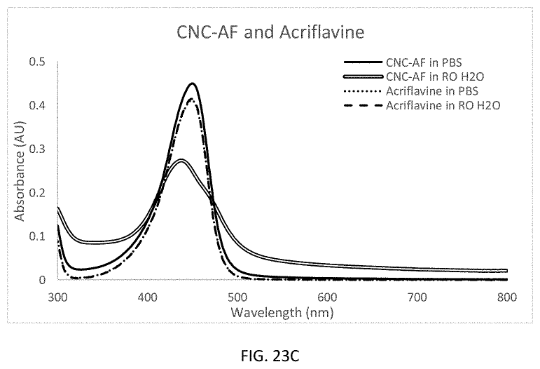

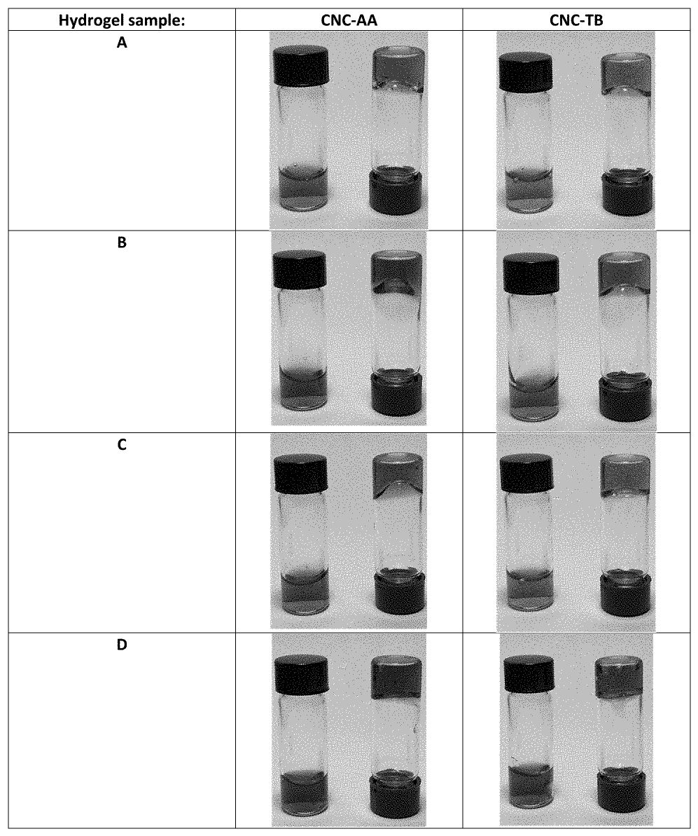

With reference to FIGS. 23A-23C, a CNC conjugate comprising the dye acriflavine (AF) was synthesized and characterized spectroscopically. The structure of acriflavine is shown in FIGS. 23A and 23B. Initial experiments suggest that the dye might not have coupled to the surface of CNC as no major shift in the absorption was observed (FIG. 23C). This could be due to the presence of a methyl group on the cationic central amine.

### REFERENCES

The following references are each individually incorporated by reference herein in their entirety:
1. Organization, W. H., Fact Sheet on Antimicrobial Resistance. 2018.
2. Cecchini, M.; Langer, J.; Slawomirski, L., Antimicrobial Resistance in G7 Countries and Beyond: Economic Issues, Policies and Options for Action. OECD, Ed. 2015.
3. Costerton, J. W.; Stewart, P. S.; Greenberg, E. P., Bacterial Biofilms: A Common Cause of Persistent Infections. Science 1999, 284 (5418), 1318.
4. Hall-Stoodley, L.; Costerton, J. W.; Stoodley, P., Bacterial biofilms: from the Natural environment to infectious diseases. Nature Reviews Microbiology 2004, 2, 95.
5. Taylor, G.; Gravel, D.; Matlow, A.; Embree, J.; LeSaux, N.; Johnston, L.; Suh, K. N.; John, M.; Embil, J.; Henderson, E.; Roth, V.; Wong, A.; Resistance, t. C. N. I. S. P. J. A.; Control, I., Assessing the magnitude and trends in hospital acquired infections in Canadian hospitals through sequential point prevalence surveys. 2016, 5 (1), 19.
6. O'Neill, J., Tackling Drug-Resistant Infections Globally: Final Report and Recommendations. 2016.
7. CDC, Antibiotic Resistance threats in the United States. 2013.
8. Strausbaugh, L. J.; Crossley, K. B.; Nurse, B. A.; Thrupp, L. D., Antimicrobial Resistance in Long-Term-Care Facilities. Infection Control & Hospital Epidemiology 1996, 17 (2), 129-140.
9. Verraes, C.; Van Boxstael, S.; Van Meervenne, E.; Van Coillie, E.; Butaye, P.; Catry, B.; de Schaetzen, M.-A.; Van Huffel, X.; Imberechts, H.; Dierick, K.; Daube, G.; Saegerman, C.; De Block, J.; Dewulf, J.; Herman, L., Antimicrobial Resistance in the Food Chain: A 25 Review. 2013, 10 (7), 2643.
10. Lindeman, C.; Leal, J.; Rusk, A.; Bush, K.; Simmonds, K.; Uma Chandran, A.; Henderson, E., Clostridium difficile infection in Alberta's long-term care facilities Corresponding Author. 2017; Vol. 32, p 87-92.
11. André, S.; Vallaeys, T.; Planchon, S., Spore-forming bacteria responsible for food spoilage. Res Microbiol 2017, 168 (4), 379-387.
12. Wainwright, M.; Crossley, K. B., Photosensitising agents—circumventing resistance and breaking down biofilms: a review. International Biodeterioration & Biodegradation 2004, 53 (2), 119-126.
13. Wainwright, M.; Maisch, T.; Nonell, S.; Plaetzer, K.; Almeida, A.; Tegos, G. P.; Hamblin, M. R., Photoantimicrobials-are we afraid of the light? The Lancet. Infectious diseases 2017, 17 (2), e49-e55.
14. Abrahamse, H.; Hamblin, Michael R., New photosensitizers for photodynamic therapy. Biochemical Journal 2016, 473 (4), 347.
15. Macia, N.; Heyne, B., Using photochemistry to understand and control the production of reactive oxygen species in biological environments. J. Photochem. Photobiol., A 2015, 306, 1-12.
16. De, S. L.; Butt, M. A.; Kohoutova, D.; Lovat, L. B.; Pye, H.; Mosse, C. A.; Yahioglu, G.; Stamati, I.; Deonarain, M.; Battah, S.; Ready, D.; Allan, E.; Mullany, P., Development of Photodynamic Antimicrobial Chemotherapy (PACT) for Clostridium difficile. PLoS One 2015, 10 (8), e0135039.
17. Eichner, A.; Gonzales, F. P.; Felgentraeger, A.; Regensburger, J.; Holzmann, T.; Schneider-Brachert, W.; Baeumler, W.; Maisch, T., Dirty hands: photodynamic killing of human pathogens like EHEC, MRSA and Candida within seconds. Photochem. Photobiol. Sci. 2013, 12 (1), 135-147.
18. Ogilby, P. R., Singlet oxygen: there is indeed something new under the sun. Chem. Soc. Rev. 2010, 39 (8), 3181-3209.
19. Lobdell, K. W.; Stamou, S.; Sanchez, J. A., Hospital-acquired infections. Surg Clin North Am 2012, 92 (1), 65-77.
20. Kampf, G.; Degenhardt, S.; Lackner, S.; Jesse, K.; von, B. H.; Ostermeyer, C., Poorly processed reusable surface disinfection tissue dispensers may be a source of infection. BMC Infect Dis 2014, 14, 37.
21. Vickery, K.; Deva, A.; Jacombs, A.; Allan, J.; Valente, P.; Gosbell, I. B., Presence of biofilm containing viable multiresistant organisms despite terminal cleaning on clinical surfaces in an intensive care unit. J Hosp Infect 2012, 80 (1), 52¬5.
22. Otter, J. A.; Vickery, K.; Walker, J. T.; deLancey, P. E.; Stoodley, P.; Goldenberg, S. D.; Edgeworth, J. D.; Salkeld, J. A. G.; Chewins, J.; Yezli, S., Surface-attached cells, biofilms and biocide susceptibility: implications for hospital cleaning and disinfection. J Hosp Infect 2015, 89 (1), 16-27.
23. Klemm, D.; Kramer, F.; Moritz, S.; Lindstrom, T.; Ankerfors, M.; Gray, D.; Dorris, A., Nanocelluloses: A New Family of Nature-Based Materials. 2011, 50 (24), 5438-5466.
24. Moon, R. J.; Martini, A.; Nairn, J.; Simonsen, J.; Youngblood, J., Cellulose nanomaterials review: structure, properties and nanocomposites. Chemical Society Reviews 2011, 40 (7), 3941-3994.
25. Dong, S.; Hirani, A. A.; Colacino, K. R.; Lee, Y. W.; Roman, M., cytotoxicity and cellular uptake of cellulose nanocrystals. Nano LIFE 2012, 02 (03), 1241006.
26. Roman, M., Toxicity of Cellulose Nanocrystals: A Review. Industrial Biotechnology 2015, 11 (1), 25-33.
27. Chen, J.; Cesario, T. C.; Rentzepis, P. M., Time resolved spectroscopic studies of methylene blue and phenothiazine derivatives used for bacteria inactivation. Chem. Phys. Lett. 2010, 498 (1-3), 81-85.
28. Vecchio, D.; Gupta, A.; Huang, L.; Landi, G.; Avci, P.; Rodas, A.; Hamblin, M. R., Bacterial photodynamic inactivation mediated by methylene blue and red light is enhanced by synergistic effect of potassium iodide. Antimicrob. Agents Chemother. 2015, 59 (9), 5203-5212.
29. Sharman, W. M.; Allen, C. M.; Van Lier, J. E., Photodynamic therapeutics: Basic principles and clinical applications. Drug Discovery Today 1999, 4 (11), 507-517.
30. Sikka, P.; Bindra, V. K.; Kapoor, S.; Jain, V.; Saxena, K. K., Blue cures blue but be cautious. Journal of pharmacy & bioallied sciences 2011, 3 (4), 543-545.
31. Wainwright, M.; Amaral, L., Review: The phenothiazinium chromophore and the evolution of antimalarial drugs. Tropical Medicine & International Health 2005, 10 (6), 501-511.
32. Wainwright, M.; Mohr, H.; Walker, W. H., Phenothiazinium derivatives for pathogen inactivation in blood products. Journal of Photochemistry and Photobiology B: Biology 2007, 86 (1), 45-58.
33. de Oliveira, B. P.; Aguiar, C. M.; Câmara, A. C., Photodynamic therapy in combating the causative microorganisms from endodontic infections. European journal of dentistry 2014, 8 (3), 424-430.
34. Klepac-Ceraj, V.; Patel, N.; Song, X.; Holewa, C.; Patel, C.; Kent, R.; Amiji, M. M.; Soukos, N. S., Photodynamic effects of methylene blue-loaded polymeric nanoparticles on dental plaque bacteria. Lasers Surg. Med. 2011, 43 (7), 600-606.
35. Spencer, W.; Sutter, J. R., Kinetic study of the monomer-dimer equilibrium of methylene blue in aqueous solution. J. Phys. Chem. 1979, 83 (12), 1573-6.
36. Vara, J.; Ortiz, C. S., Thiazine dyes: Evaluation of monomeric and aggregate forms. Spectrochimica Acta Part A: Molecular and Biomolecular Spectroscopy 2016, 166, 112-120.
37. Faure, J.; Bonneau, R.; Joussot-Dubien, J., Etude en Spectroscopie par Eclair des Colorants Thiaziniques en Solution Aqueuse. Photochem. Photobiol. 1967, 6 (5), 331-339.
38. Havelcové, M.; Kubát, P.; Němcová, I., Photophysical properties of thiazine dyes in aqueous solution and in micelles. Dyes and Pigments 1999, 44 (1), 49-54.
39. Macia, N.; Bresoli-Obach, R.; Nonell, S.; Heyne, B., Hybrid Silver Nanocubes for Improved Plasmon-Enhanced Singlet Oxygen Production and Inactivation of Bacteria. Journal of the American Chemical Society 2019, 141 (1), 684-692.
40. Planas, O.; Macia, N.; Agut, M.; Nonell, S.; Heyne, B., Distance-Dependent Plasmon-Enhanced Singlet Oxygen Production and Emission for Bacterial Inactivation. J. Am. Chem. Soc. 2016, 138 (8), 2762-2768.
41. Nonell, S.; Flors, C., Chapter 25 Steady-State and Time-Resolved Singlet Oxygen Phosphorescence Detection in the Near-IR. In Singlet Oxygen: Applications in Biosciences and Nanosciences, Volume 2, The Royal Society of Chemistry: 2016; Vol. 2, pp 7-26.
42. Goldman, E.; Green, L. H., Practical handbook of microbiology. 2nd ed. ed.; Boca Raton: CRC Press: Boca Raton, 2009.
43. Miyoshi-Akiyama, T.; Tada, T.; Ohmagari, N.; Viet Hung, N.; Tharavichitkul, P.; Pokhrel, B. M.; Gniadkowski, M.; Shimojima, M.; Kirikae, T., Emergence and Spread of Epidemic Multidrug-Resistant Pseudomonas aeruginosa. Genome biology and evolution 2017, 9 (12), 3238-3245.
44. Navon-Venezia, S.; Kondratyeva, K.; Carattoli, A., Klebsiella pneumoniae: a major worldwide source and shuttle for antibiotic resistance. FEMS Microbiology Reviews 2017, 41 (3), 252-275.
45. Potron, A.; Poirel, L.; Nordmann, P., Emerging broad-spectrum resistance in Pseudomonas aeruginosa and Acinetobacter baumannii: Mechanisms and epidemiology. International Journal of Antimicrobial Agents 2015, 45 (6), 568-585.
46. Vuotto, C.; Longo, F.; Pascolini, C.; Donelli, G.; Balice, M. P.; Libori, M. F.; Tiracchia, V.; Salvia, A.; Varaldo, P. E., Biofilm formation and antibiotic resistance in Klebsiella pneumoniae urinary strains. Journal of Applied Microbiology 2017, 123 (4), 1003-1018.
47. Li, X.-Z.; Plésiat, P.; Nikaido, H., The Challenge of Efflux-Mediated Antibiotic Resistance in Gram-Negative Bacteria. Clinical Microbiology Reviews 2015, 28 (2), 337.
48. Lister, P. D.; Wolter, D. J.; Hanson, N. D., Antibacterial-resistant Pseudomonas aeruginosa: clinical impact and complex regulation of chromosomally encoded resistance mechanisms. Clinical microbiology reviews 2009, 22 (4), 582-610.
49. Poole, K., Efflux-mediated antimicrobial resistance. Journal of Antimicrobial Chemotherapy 2005, 56 (1), 20-51.
50. Tegos, G. P.; Masago, K.; Aziz, F.; Higginbotham, A.; Stermitz, F. R.; Hamblin, M. R., Inhibitors of bacterial multidrug efflux pumps potentiate antimicrobial photoinactivation. Antimicrobial agents and chemotherapy 2008, 52 (9), 3202-3209.
51. Kasimova, K. R., Sadasivam, M., Landi, G., Sarna, T., & Hamblin, M. R. (2014). Potentiation of photoinactivation of Gram-positive and Gram-negative bacteria mediated by six phenothiazinium dyes by addition of azide ion. Photochem. Photobiol. Sci., 13(11), 1541-1548.
52. Ronzani, F., Trivella, A., Arzoumanian, E., Blanc, S., Sarakha, M., Richard, C., Lacombe, S. (2013). Comparison of the photophysical properties of three phenothiazine derivatives: transient detection and singlet oxygen production. Photochemical & Photobiological Sciences, 12(12), 2160.
53. Mohr, H., Bachmann, B., Klein Struckmeier, A., & Lambrecht, B. (1996). Virus Inactivation of Blood Products by Phenothiazine Dyes and Light. Photochemistry and Photobiology, 65(3), 441-445.
54. Stanley, S. L., Scholle, F., Zhu, J., Lu, Y., Zhang, X., Situ, X., & Ghiladi, R. A. (2016). Photosensitizer-Embedded Polyacrylonitrile Nanofibers as Antimicrobial Non-Woven Textile. Nanomaterials, 6(4), 77.
55. Leng, T., Jakubek, Z. J., Mazloumi, M., Leung, A. C. W., & Johnston, L. J. (2017). Ensemble and Single Particle Fluorescence Characterization of Dye-Labeled Cellulose Nanocrystals. Langmuir, 33(32), 8002— 8011.
56. Dufresne, A. (2013). Nanocellulose: a new ageless bionanomaterial. Materials Today, 16(6), 220-227.
57. Mann, E. E.; Wozniak, D. J. (2012). Pseudomonas biofilm matrix composition and niche biology. FEMS Microbiol Rev., 36(4): 893-916.
58. Harmsen, M.; Yang, L.; Pamp, S. J.; Tolker-Nielsen, T. (2010) An update on Pseudomonas aeruginosa biofilm formation, tolerance, and dispersal. FEMS Immunol Med Microbiol., 59,253-268.
59. James, G. A.; Swogger, E.; Wolcott, R.; deLancey Pulcini, E.; Secor, P.; Sestrich, J.; Costerton, J. W.; Stewart, P. S. (2008) Biofilms in chronic wounds, 16(1), 37-44.
60. Bjarnsholt, T.; Kirketerp-Moller, K.; Ostrup Jensen, P.; Madsen, K. G.; Phipps, R.; Krogfelt, K.; Hoiby, N.; Givskov, M. (2008) Why chronic wounds will not heal: a novel hypothesis, Wound Rep. Reg., 16,2-10.
61. Gjodsbol, K.; Christensen, J. J.; Karlsmaek, T.; Jorgensen, B.; Klein, B. M.; Krogfelt, K. A. (2006) Multiple bacterial species reside in chronic wounds: a longitudinal study. Int. Wound J., 3(3), 225-231.
62. Harrison, J. J.; Turner, R. J.; Ceri, H. (2005) Persister cells, the biofilm matrix and tolerance to metal cations in biofilm and planktonic Pseudomonas aeruginosa. Env. Microbiol., 7(7), 981-994.
63. Muller, M. (1995) Scavenging of neutrophil derived superoxide anion by 1-hydroxyphenazine, a phenazine derivative associated with chronic Pseudomonas aeruginosa infection: Relevance to cystic fibrosis. Biochim. Biophys. Acta 1272(3), 185-189
64. Simpson, J. A.; Smith, S. E.; Dean, R. T. (1989) Scavenging by alginate of free radicals released by macrophages. Free Radic. Biol. Med. 6(4), 347-353.
65. Street, C. N.; Gibbs, A.; Pedigo, L; Andersen, D.; Loebel, N. G. (2009) In Vitro photodynamic eradication of Pseudomonas aeruginosa in Planktonic and Biofilm Culture. Photochem. Photobiol., 85, 137-143.
66. Sannino, A.; Demitry, C.; Mafaghiele, M. (2009) Biodegradable Cellulose-based hydrogels: design and applications. Materials, 2, 353-373.
67. Calo, E.; Khutoryanskiy, V. V. (2015) Biomedical applications of hydrogels: A review of patents and commercial products. Eur. Polymer J., 65, 252-267.

1. A composition comprising:
(a) cellulose nanocrystals (CNCs); and
(b) photosensitizer molecules conjugated to the CNCs,
wherein the photosensitizer molecules are adsorbed to the CNCs;
wherein the composition generates reactive singlet oxygen when exposed to light.

2. (canceled)

3. (canceled)

4. (canceled)

5. (canceled)

6. (canceled)

7. (canceled)

8. The composition of any claim 1, wherein the concentration of photosensitizer molecules in the composition is within the range of 0.03 to 0.075 (mmol/100 mg).

9. The composition of claim 1, wherein the photosensitizer molecules are selected from the group consisting of a cationic dye with acidic proton, azure A, azure B, toluidine blue O, thionine acetate and cresyl violet.

10. The composition of claim 9, wherein the photosensitizer molecules are azure A.

11. (canceled)

12. The composition of claim 1, wherein the photosensitizer molecules each have a substantially planar conformation.

13. A method of preparing the composition of claim 1 comprising:
(a) providing a suspension of oxidized CNCs having an acidic pH;
(b) dispersing the photosensitizer molecules in the suspension;
(c) modifying the pH of the suspension to an alkaline pH to cause the photosensitizer molecules to adsorb to the CNCs to form the composition; and
(d) acidifying the suspension to yield a stable form of the composition.

14. The method of claim 13, wherein the alkaline pH is within the range of approximately 10-11.

15. The method of claim 13, comprising maintaining the suspension at the alkaline pH for at least 16 hours prior to acidifying the suspension.

16. The method of claim 13, wherein the oxidation level of the CNCs exceeds 0.75 mmol of CO₂H/gram.

17. The method of claim 16, wherein the oxidation level of the CNCs is within the range of 1-1.5 mmol of CO₂H/gram.

18. The method of claim 13, wherein the acidifying comprises adding HCl to the suspension to adjust the pH to approximately 1.

19. A photobiocidal disinfectant formulation comprising the composition of claim 1 and an aqueous-compatible media.

20. (canceled)

21. The formulation of claim 19, wherein the media is a film-forming polymer, a hydrogel, or an aqueous solvent.

22. (canceled)

23. (canceled)

24. The formulation of claim 20, wherein the photobiocidal disinfectant formulation is for disinfecting wounds and the media is a biocompatible hydrogel.

25. The formulation of claim 24, wherein the concentration of the composition in the formulation is 0.01 to 10% by wt/v.

26. (canceled)

27. (canceled)

28. A method of disinfecting a surface comprising:
(a) applying the formulation of claim 19 to the surface; and
(b) activating the photosensitizer molecules by applying light to the surface, thereby causing the composition to generate reactive singlet oxygen.

29. The method of claim 28, wherein the media is a biocompatible hydrogel, and wherein the surface is a wound.

30. The method of claim 28, wherein the surface is comprised of a material selected from the group consisting of glass, wood, wood composite, rubber, vinyl, tile, PVS, ABS, polyethylene, nylon, steel, brass, nickel, copper, zinc and aluminum.

31. The method of claim 28, wherein the light has a wavelength within the range of the visible spectrum.

32. (canceled)

33. (canceled)

34. (canceled)

35. (canceled)

36. A medical device comprising the photobiocidal disinfectant formulation of claim 24.

37. The method of claim 13, wherein providing a suspension of oxidized CNCs having an acidic pH comprises:
i) functionalizing the surface of CNCs; and
ii) oxidizing functional groups on the surface of the CNCs to provide the suspension of oxidized CNCs having an acidic pH.

38. (canceled)

39. The method of in claim 37, wherein the functionalizing comprises carboxylation.

40. (canceled)

41. (canceled)

42. (canceled)

43. The method of claim 28, wherein photobactericidal potency of the composition or conjugate is more than twice as toxic to bacteria than the light-activated photosensitizer molecules in a non-conjugated free form.

44. (canceled)

45. (canceled)

46. (canceled)
